(19) United States
(12) Patent Application Publication
(10) Pub. No.: US 2009/0142836 A1
(43) Pub. Date: Jun. 4, 2009

(54) BIOENGINEERED TISSUE CONSTRUCTS AND METHODS FOR PRODUCTION AND USE

(75) Inventors: Xianyan Wang, Acton, MA (US); Katherine C. Faria, Middleboro, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Organogenesis, Inc., Canton, MA (US)

(21) Appl. No.: 12/324,367

(22) Filed: Nov. 26, 2008

Related U.S. Application Data
(60) Provisional application No. 60/990,757, filed on Nov. 28, 2007.
(60) Provisional application No. 61/021,176, filed on Jan. 15, 2008.

(51) Int. Cl. C12N 5/06

(52) U.S. Cl. 435/395

(57) ABSTRACT

Bioengineered constructs are formed from cultured cells induced to synthesize and secrete endogenously produced extracellular matrix components without the requirement of exogenous matrix components or network support or scaffold members. The bioengineered constructs of the invention can be treated in various ways such that the cells of the bioengineered constructs can be devitalized and/or removed without compromising the structural integrity of the constructs. Moreover, the bioengineered constructs of the invention can be used in conjunction with biocompatible/bioremodelable solutions that allow for various geometric configurations of the constructs.

### RELATED APPLICATIONS

This application claims the benefit of U.S. Provisional Application Ser. No. 60/990,757, filed on Nov. 28, 2007 and of U.S. Provisional Application Ser. No. 61/021,176, filed on Jan. 15, 2008; the entire contents of each of the applications is incorporated herein by reference.

### FIELD OF THE INVENTION

The invention is in the field of tissue engineering. This invention is directed to a method for producing a bioengineered constructs. This bioengineered constructs are biocompatible and bioremodelable and can be used for clinical purposes.

### BACKGROUND OF THE INVENTION

The subject invention relates to disciplines of tissue engineering, tissue regeneration and regenerative medicine combines bioengineering methods with the principles of life sciences to understand the structural and functional relationships in normal and pathological mammalian tissues. The overall goal of these disciplines is the development and ultimate application of biological substitutes to restore, maintain, or improve tissue functions. Thus, it is possible to design and manufacture a bioengineered tissue in a laboratory. Bioengineered tissues can include cells that are usually associated with a native mammalian or human tissues and synthetic or natural matrix scaffolds. The new bioengineered tissue must be functional when grafted onto a host, and be permanently incorporated within the host's body or progressively bioremodeled by cells from the bioengineered tissue or recipient host. Fabrication of bioengineered tissue constructs without the incorporation or reliance on exogenous support members or scaffolds leads to scientific challenges in creating the new bioengineered tissue constructs.

### SUMMARY OF THE INVENTION

The invention is directed to bioengineered tissue constructs produced by cultured cells and endogenously produced extracellular matrix components without the requirement of exogenous matrix components or network support or scaffold members. The invention can thus advantageously be made entirely from human cells, and human matrix components produced by those cells, for example, when the bioengineered tissue construct is designed for use in humans.

The invention is also directed to methods for producing tissue constructs by stimulation of cells in culture, such as fibroblasts, to produce extracellular matrix components without the addition of either exogenous matrix components, network support, or scaffold members.

The invention is also directed to methods for producing tissue constructs by stimulation of cells in culture, such as fibroblasts, to produce extracellular matrix components in a defined medium system and/or without the use of undefined or non-human-derived biological components, such as bovine serum or organ extracts.

Further, the invention is directed to bioengineered constructs comprising a devitalized and/or decellularized layer of extracellular matrix produced and assembled by cultured extracellular matrix producing cells.

Still further, the invention is directed towards a method for making a bioengineered construct, comprising producing two or more layers of endogenously produced extracellular matrices and subsequently devitalizing and or decellularizing the extracellular matrix producing cells prior to combining the two or more layers via crosslinking and/or biocompatible and bioremodelable adhesive solutions.

tissue construct is produced and self-assembled by cultured cells without the need for scaffold support or the addition of exogenous extracellular matrix components.

### DETAILED DESCRIPTION OF THE INVENTION

Heretofore, current engineered living tissue constructs are not completely cell assembled and must rely on either the addition or incorporation of exogenous matrix components or synthetic members for structure or support, or both.

The bioengineered tissue constructs described herein exhibit many of the native features of the tissue from which their cells are derived. The tissue constructs thus produced can be used for grafting to a subject or for in vitro testing.

One preferred embodiment is a cell-matrix construct comprising a first cell type and endogenously produced extracellular matrix wherein the first cell type is capable of synthesizing and secreting extracellular matrix to produce the cell-matrix construct.

Another preferred embodiment is a bilayer construct comprising a first cell type and endogenously produced extracellular matrix and a layer of cells of a second type disposed thereon or within the cell-matrix construct formed by the first cell type.

A more preferred embodiment is a cell-matrix construct comprising fibroblasts, such as those derived dermis, to form a cultured dermal construct.

Another more preferred embodiment is a cell-matrix construct comprising fibroblasts, such as those derived from dermis, to form a cultured dermal construct with a layer of keratinocytes cultured thereon to form an epidermal layer to result in a cultured bilayer skin construct. The cultured skin constructs of the invention express many physical, morphological, and biochemical features of native skin.

In an even more preferred embodiment, the cell-matrix construct is a tissue construct that is similar to the dermal layer of skin, a human dermal construct, that is formed in a defined system comprising human-derived cells utilizing no chemically undefined components during its culture.

In the most preferred embodiment, the tissue constructs of the invention are fabricated in a chemically defined system comprising human-derived cells but no chemically undefined or non-human biological components or cells.

One preferred embodiment of the invention comprises a structural layer of at least one type of extracellular matrix-producing cells and endogenously produced extracellular matrix components, more simply termed “matrix”, wherein the matrix is completely cell-synthesized and assembled by culturing the cells. This layer is herein termed a “cell-matrix construct” or a “cell-matrix layer” because the cells secrete and contain themselves within and through their matrix. The cultured tissue constructs do not require, thus do not include, exogenous matrix components, that is, matrix components not produced by the cultured cells but introduced by other means. In a more preferred embodiment, the cell-matrix construct produced by human dermal fibroblasts is shown to have a predominant concentration of collagen similar to native skin. As evidenced by electron microscopy, the matrix is fibrous in nature comprising collagen that exhibits the quarter-staggered 67 nm banding pattern, as well as packing organization of fibrils and fibril bundles similar to native collagen. Delayed reduction SDS-PAGE has detected the presence of both type I and type III collagen in these constructs, the predominant collagen types found in native human skin. Using standard immunohistochemistry (IHC) techniques, the dermal cell-matrix construct stains positively for decorin, a dermatan sulfate proteoglycan known to be associated with collagen fibrils and believed to regulate fibril diameter in vivo. Decorin can also be visualized in the construct with TEM. The produced tissue also stains positive for tenascin, an extracellular matrix glycoprotein found, for example, in mesenchyme or tissues under repair. Much like tissue under repair in vivo, the tissue produced in culture has been shown to increase its ratio of type I to type III collagen as the matrix is formed. While not wishing to be bound by theory, it is believed that the cells fill in the open space between them quickly with a loose matrix analogous to granulation tissue comprised of mostly type III collagen and fibronectin, and then remodel this loose matrix with a denser matrix comprised of mostly type I collagen. The produced cell-matrix has been shown to contain glycosaminoglycans (GAG), such as hyaluronic acid (HA); fibronectin; proteoglycans besides decorin such as biglycan and versican; and, a profile of sulfated glycosaminoglycans such as di-hyaluronic acid; di-chondroitin-O-sulfate; di-chondroitin-4-sulfate; di-chondroitin-6-sulfate; di-chondroitin-4,6-sulfate; di-chondroitin-4-sulfate-UA-2S; and di-chondroitin-6-sulfate-UA-2S. These structural and biochemical features exhibit themselves as the construct develops in culture and are distinctively evident when the construct approaches its final form. The presence of these components in fully formed cultured dermal cell-matrix construct indicates that the construct has structural and biochemical features approaching that of normal dermis.

While the aforementioned list is a list of biochemical and structural features a cultured cell-matrix construct formed from dermal fibroblasts, it should be recognized that cultured cell-matrix constructs formed from other types of fibroblasts will produce many of these features and others phenotypic for tissue type from which they originated. In some cases, fibroblasts can be induced to express non-phenotypic components by either chemical exposure or contact, physical stresses, or by transgenic means. Another preferred embodiment of the invention is a cell-matrix layer having second layer of cells disposed thereon. The second layer of cells is cultured on the cell-matrix layer to form a bioengineered bilayered tissue construct. In a more preferred embodiment, the cells of the second layer are of epithelial origin. In the most preferred embodiment, the second layer comprises cultured human keratinocytes that together with a first cell-matrix layer, a cell-matrix construct formed from dermal fibroblasts and endogenous matrix to form a dermal layer, comprise a living skin construct. When fully formed, the epidermal layer is a multilayered, stratified, and well-differentiated layer of keratinocytes that exhibit a basal layer, a suprabasal layer, a granular layer and a stratum corneum. The skin construct has a well-developed basement membrane present at the dermal-epidermal junction as exhibited by transmission electron microscopy (TEM). The basement membrane appears thickest around hemidesmosomes, marked by anchoring fibrils that are comprised of type VII collagen, as visualized by TEM. The anchoring fibrils can seen exiting from the basement membrane and entrapping the collagen fibrils in the dermal layer. These anchoring fibrils, as well as other basement membrane components, are secreted by keratinocytes. It is also known that while keratinocytes are capable of secreting basement membrane components on their own, a recognizable basement membrane will not form in the absence of fibroblasts. Immunohistochemical staining of the skin construct of the present invention has also shown that laminin, a basement membrane protein is present.

In a preferred method of the invention for forming a cell-matrix construct, a first cell type, an extracellular matrix-producing cell type, is seeded to a substrate, cultured, and induced to synthesize and secrete an organized extracellular matrix around them to form a cell-matrix construct. In another preferred method of the invention, a surface of the cell-matrix construct is seeded with cells of a second cell type and are cultured to form bilayered tissue construct. In a more preferred method, a full thickness skin construct having features similar to native human skin is formed by culturing fibroblasts, such as human dermal fibroblasts, under conditions sufficient to induce matrix synthesis to form a cell-matrix of dermal cells and matrix, a dermal layer, onto which human epithelial cells, such as keratinocytes, are seeded and cultured under conditions sufficient to form a fully differentiated stratified epidermal layer.

Therefore, one method of obtaining the bioengineered tissue constructs of the present invention comprises: (a) culturing at least one extracellular matrix-producing cell type in the absence of exogenous extracellular matrix components or a structural support member; (b) stimulating the cells of step (a) to synthesize, secrete, and organize extracellular matrix components to form a tissue-construct comprised of cells and matrix synthesized by those cells; wherein steps (a) and (b) may be done simultaneously or consecutively; and, (c) devitalizing or decellularizing the tissue construct comprising extracellular matrix components for clinical use. Two or more devitalized or decellularized tissue constructs may be contacted together and bonded together either by way of crosslinking or the use of a biocompatible or bioresorbable adhesive.

### I. Media Formulations

Cell-matrix constructs are formed by culturing cells in a culture medium that promotes cell viability, proliferation and synthesis of extracellular matrix components by the cells. Culture medium is comprised of a nutrient base usually further supplemented with other components. The skilled artisan can determine appropriate nutrient bases in the art of animal cell culture with reasonable expectations for successfully producing a tissue construct of the invention. Many commercially available nutrient sources are useful on the practice of the present invention. These include commercially available nutrient sources which supply inorganic salts, an energy source, amino acids, and B-vitamins such as Dulbecco's Modified Eagle's Medium (DMEM); Minimal Essential Medium (MEM); M199; RPMI 1640; Iscove's Modified Dulbecco's Medium (EDMEM). Minimal Essential Medium (MEM) and M199 require additional supplementation with phospholipid precursors and non-essential amino acids. Commercially available vitamin-rich mixtures that supply additional amino acids, nucleic acids, enzyme cofactors, phospholipid precursors, and inorganic salts include Ham's F-12, Ham's F-10, NCTC109, and NCTC 135. Albeit in varying concentrations, all basal media provide a basic nutrient source for cells in the form of glucose, amino acids, vitamins, and inorganic ions, together with other basic media components. The most preferred base medium of the invention comprises a nutrient base of either calcium-free or low calcium Dulbecco's Modified Eagle's Medium (DMEM), or, alternatively, DMEM and Ham's F-12 between a 3-to-1 ratio to a 1-to-3 ratio, respectively.

The base medium is supplemented with components such as amino acids, growth factors, and hormones. Defined culture media for the culture of cells of the invention are described in U.S. Pat. No. 5,712,163 to Parenteau, International PCT Publication No. WO 95/31473, and PCT Publication No. WO 00/29553 the disclosures of which are incorporated herein by reference. Other media are known in the art such as those disclosed in Ham and McKeehan, Methods in Enzymology, 58:44-93 (1979), or for other appropriate chemically defined media, in Bottenstein et al., Methods in Enzymology, 58:94-109 (1979). In the preferred embodiment, the base medium is supplemented with the following components known to the skilled artisan in animal cell culture: insulin, transferrin, triiodothyronine (T3), and either or both ethanolamine and o-phosphoryl-ethanolamine, wherein concentrations and substitutions for the supplements may be determined by the skilled artisan.

Culture media formulations suitable for use in the present invention are selected based on the cell types to be cultured and the tissue structure to be produced. The culture medium that is used and the specific culturing conditions needed to promote cell growth, matrix synthesis, and viability will depend on the type of cell, or combinations of types of cells, being grown.

In some instances, such as in the fabrication of bioengineered bilayer skin constructs of the present invention, the media composition varies with each stage of fabrication as different supplementation is necessary for different purposes. In a preferred method, the cell-matrix layer is formed under defined conditions, that is, cultured in chemically defined media. In another preferred method, a tissue construct comprises a cell-matrix layer provided with a second layer of cells disposed and cultured thereon wherein both cell types are cultured in a defined culture media system. Alternatively, the tissue construct comprises a cell-matrix layer fabricated under defined media conditions and a second layer formed thereon under undefined media conditions. In the converse, the tissue construct comprises a cell-matrix layer may be fabricated under undefined media conditions and the second layer formed thereon under defined media conditions.

The use of chemically defined culture media is preferred, that is, media free of undefined animal organ or tissue extracts, for example, serum, pituitary extract, hypothalamic extract, placental extract, or embryonic extract or proteins and factors secreted by feeder cells. In a most preferred embodiment, the media is free of undefined components and biological components derived from non-human animal sources. Although the addition of undefined components is not preferred, they may be used in accordance with the disclosed methods at any point in culture in order to fabricate successfully a tissue construct. When the invention is carried out utilizing screened human cells cultured using chemically defined components derived from no non-human animal sources, the resultant tissue construct is a defined human tissue construct. Synthetic or recombinant functional equivalents may also be added to supplement chemically defined media within the purview of the definition of chemically defined for use in the most preferred fabrication method. Generally, one of skill in the art of cell culture will be able to determine suitable natural human, human recombinant, or synthetic equivalents to commonly known animal components to supplement the culture media of the invention without undue investigation or experimentation. The advantages in using such a construct in the clinic is that the concern of adventitious animal or cross-species virus contamination and infection is diminished. In the testing scenario, the advantages of a chemically defined construct is that when tested, there is no chance of the results being confounded due to the presence of the undefined components.

Insulin is a polypeptide hormone that promotes the uptake of glucose and amino acids to provide long term benefits over multiple passages. Supplementation of insulin or insulin-like growth factor (IGF) is necessary for long term culture as there will be eventual depletion of the cells' ability to uptake glucose and amino acids and possible degradation of the cell phenotype. Insulin may be derived from either animal, for example bovine, human sources, or by recombinant means as human recombinant insulin. Therefore, a human insulin would qualify as a chemically defined component not derived from a non-human biological source. Insulin supplementation is advisable for serial cultivation and is provided to the media at a wide range of concentrations. A preferred concentration range is between about 0.1 μg/ml to about 500 μg/ml, more preferably at about 5 μg/ml to about 400 μg/ml, and most preferably at about 375 μg/ml. Appropriate concentrations for the supplementation of insulin-like growth factor, such as IGF-1 IGF-2, and the like may be easily determined by one of skill in the art for the cell types chosen for culture.

Transferrin is in the medium for iron transport regulation. Iron is an essential trace element found in serum. As iron can be toxic to cells in its free form, in serum it is supplied to cells bound to transferrin at a concentration range of preferably between about 0.05 to about 50 μg/ml, more preferably at about 5 μg/ml.

Triiodothyronine (T3) is a basic component and is the active form of thyroid hormone that is included in the medium to maintain rates of cell metabolism. Triiodothyronine is supplemented to the medium at a concentration range between about 0 to about 400 ρM, more preferably between about 2 to about 200 ρM and most preferably at about 20 ρM.

Either or both ethanolamine and o-phosphoryl-ethanolamine, which are phospholipids, are added whose function is an important precursor in the inositol pathway and fatty acid metabolism. Supplementation of lipids that are normally found in serum is necessary in a serum-free medium. Ethanolamine and o-phosphoryl-ethanolamine are provided to media at a concentration range between about 10⁻⁶ to about 10⁻² M, more preferably at about 1×10⁻⁴ M.

Throughout the culture duration, the base medium is additionally supplemented with other components to induce synthesis or differentiation or to improve cell growth such as hydrocortisone, selenium, and L-glutamine.

Hydrocortisone has been shown in keratinocyte culture to promote keratinocyte phenotype and therefore enhance differentiated characteristics such as involucrin and keratinocyte transglutaminase content (Rubin et al., J. Cell Physiol., 138:208-214 (1986)). Therefore, hydrocortisone is a desirable additive in instances where these characteristics are beneficial such as in the formation of keratinocyte sheet grafts or skin constructs. Hydrocortisone may be provided at a concentration range of about 0.01 μg/ml to about 4.0 μg/ml, most preferably between about 0.4 μg/ml to 16 μg/ml.

Selenious acid is added to serum-free media to resupplement the trace elements of selenium normally provided by serum. Selenious acid may be provided at a concentration range of about 10⁻⁹ M to about 10⁻⁷ M; most preferably at about 5.3×10⁻⁸ M.

The amino acid L-glutamine is present in some nutrient bases and may be added in cases where there is none or insufficient amounts present. L-glutamine may also be provided in stable form such as that sold under the mark, GlutaMAX-1™ (Gibco BRL, Grand Island, N.Y.). GlutaMAX-1™ is the stable dipeptide form of L-alanyl-L-glutamine and may be used interchangeably with L-glutamine and is provided in equimolar concentrations as a substitute to L-glutamine. The dipeptide provides stability to L-glutamine from degradation over time in storage and during incubation that can lead to uncertainty in the effective concentration of L-glutamine in medium. Typically, the base medium is supplemented with preferably between about 1 mM to about 6 mM, more preferably between about 2 mM to about 5 mM, and most preferably 4 mM L-glutamine or GlutaMAX-1™.

Growth factors such as epidermal growth factor (EGF) may also be added to the medium to aid in the establishment of the cultures through cell scale-up and seeding. EGF in native form or recombinant form may be used. Human forms, native or recombinant, of EGF are preferred for use in the medium when fabricating a skin equivalent containing no non-human biological components. EGF is an optional component and may be provided at a concentration between about 1 to 15 ng/mL, more preferably between about 5 to 10 ng/mL.

Other supplements may also be added to the medium, such as one or more prostaglandins, transforming growth factors (including transforming growth factors alpha or beta), keratinocyte growth factor (KGF), connective tissue growth factor (CTGF), or mannose-6-phosphate (M6P), or a combination thereof.

Prostaglandin E₂ (PGE₂) is generated from the action of prostaglandin E synthases on prostaglandin H₂ (PGH₂). Several prostaglandin E synthases have been identified. To date, microsomal prostaglandin E synthase-1 emerges as a key enzyme in the formation of PGE₂. PGE₂ is supplemented to the medium preferably in the range from about 0.000038 μg/mL to about 0.760 μg/mL, more preferably from about 0.00038 μg/mL to about 0.076 μg/mL, most preferably from about 0.0038 μg/mL to about 0.038 μg/mL. The 16,16 PGE₂ form may also be supplemented in these ranges.

Transforming growth factor alpha (TGF-α) is produced in macrophages, brain cells, and keratinocytes, and induces epithelial development. It is closely related to EGF, and can also bind to the EGF receptor with similar effects. Preferably the long chain form of TGF-α is employed in the invention. TGF-alpha is a small (˜50 residue) protein that shares 30% structural homology with EGF and competes for the same surface-bound receptor site. It has been implicated in wound healing and promotes phenotypic changes in certain cells. TGF alpha or long-chain TGF alpha is supplemented to the medium preferably in the range from about 0.0005 μg/mL to about 0.30 μg/mL, more preferably from about 0.0050 μg/mL to about 0.03 μg/mL, most preferably from about 0.01 μg/mL to about 0.02 μg/mL.

Supplementation of the base medium with keratinocyte growth factor 5 μg/mL may be used to support epidermalization. Keratinocyte growth factor (KGF) is supplemented to the medium preferably in the range from about 0.001 μg/mL to about 0.150 μg/mL, more preferably from about 0.0025 μg/mL to about 0.100 μg/mL, most preferably from about 0.005 μg/mL to about 0.015 μg/mL.

Supplementation of the base medium with mannose-6-phosphate (M6P) may be used to support epidermalization Mannose-6-Phosphate is supplemented to the medium preferably in the range from about 0.0005 mg/mL to about 0.0500 mg/mL.

CTGF (connective tissue growth factor) is a cysteine-rich, matrix-associated, heparin-binding protein. In vitro, CTGF mirrors some of the effects of TGF beta on skin fibroblasts, such as stimulation of extracellular matrix production, chemotaxis, proliferation and integrin expression. CTGF can promote endothelial cell growth, migration, adhesion and survival and is thus implicated in endothelial cell function and angiogenesis. CTGF binds to perlecan, a proteoglycan which has been localised in synovium, cartilage and numerous other tissues. CTGF has been implicated in extracellular matrix remodeling in wound healing, scleroderma and other fibrotic processes, as it is capable of upregulating both matrix metalloproteinases (MMPs) and their inhibitors (TIMPs). Therefore, CTGF has the potential to activate both the synthesis and degradation of the extracellular matrix.

The medium described above is typically prepared as set forth below. However, it should be understood that the components of the present invention may be prepared and assembled using conventional methodology compatible with their physical properties. It is well known in the art to substitute certain components with an appropriate analogous or functionally equivalent acting agent for the purposes of availability or economy and arrive at a similar result. Naturally occurring growth factors may be substituted with recombinant or synthetic growth factors that have similar qualities and results when used in the performance of the invention.

Media in accordance with the present invention are sterile. Sterile components are bought sterile or rendered sterile by conventional procedures, such as filtration, after preparation. Proper aseptic procedures were used throughout the following Examples. DMEM and F-12 are first combined and the individual components are then added to complete the medium. Stock solutions of all components can be stored at −20° C., with the exception of nutrient source that can be stored at 4° C. All stock solutions are prepared at 500× final concentrations listed above. A stock solution of insulin, transferrin and triiodothyronine (all from Sigma) is prepared as follows: triiodothyronine is initially dissolved in absolute ethanol in 1N hydrochloric acid (HCl) at a 2:1 ratio. Insulin is dissolved in dilute HCl (approximately 0.1N) and transferrin is dissolved in water. The three are then mixed and diluted in water to a 500× concentration. Ethanolamine and o-phosphoryl-ethanolamine are dissolved in water to 500× concentration and are filter sterilized. Progesterone is dissolved in absolute ethanol and diluted with water. Hydrocortisone is dissolved in absolute ethanol and diluted in phosphate buffered saline (PBS). Selenium is dissolved in water to 500× concentration and filter sterilized. EGF is purchased sterile and is dissolved in PBS. Adenine is difficult to dissolve but may be dissolved by any number of methods known to those skilled in the art. Serum albumin may be added to certain components in order to stabilize them in solution and are presently derived from either human or animal sources. For example, human serum albumin (HSA) or bovine serum albumin (BSA) may be added for prolonged storage to maintain the activity of the progesterone and EGF stock solutions. Recombinant forms of albumin have been developed, such as a human recombinant albumin, and their substitution instead of human and bovine serum-derived forms is preferred. The medium can be either used immediately after preparation or, stored at 4° C. If stored, EGF should not be added until the time of use.

In order to form the cell-matrix layer by the culture of matrix-producing cells, the medium is supplemented with additional agents that promote matrix synthesis and deposition by the cells. These supplemental agents are cell-compatible, defined to a high degree of purity and are free of contaminants. The medium used to produce the cell-matrix layer is termed “matrix production medium”.

To prepare the matrix production medium, the base medium is supplemented with an ascorbate derivative such as sodium ascorbate, ascorbic acid, or one of its more chemically stable derivatives such as L-ascorbic acid phosphate magnesium salt n-hydrate. Ascorbate is added to promote hydroxylation of proline and secretion of procollagen, a soluble precursor to deposited collagen molecules. Ascorbate has also been shown to be an important cofactor for post-translational processing of other enzymes as well as an upregulator of type I and type III collagen synthesis.

While not wishing to be bound by theory, supplementing the medium with amino acids involved in protein synthesis conserves cellular energy by not requiring the cells produce the amino acids themselves. The addition of proline and glycine is preferred as they, as well as the hydroxylated form of proline, hydroxyproline, are basic amino acids that make up the structure of collagen.

While not required, the matrix-production medium is optionally supplemented with a neutral polymer. The cell-matrix constructs of the invention may be produced without a neutral polymer, but again not wishing to be bound by theory, its presence in the matrix production medium may collagen processing and deposition more consistently between samples. One preferred neutral polymer is polyethylene glycol (PEG), which has been shown to promote in vitro processing of the soluble precursor procollagen produced by the cultured cells to matrix deposited collagen. Tissue culture grade PEG within the range between about 1000 to about 4000 MW (molecular weight), more preferably between about 3400 to about 3700 MW is preferred in the media of the invention. Preferred PEG concentrations are for use in the method may be at concentrations at about 5% w/v or less, preferably about 0.01% w/v to about 0.5% w/v, more preferably between about 0.025% w/v to about 0.2% w/v, most preferably about 0.05% w/v. Other culture grade neutral polymers such dextran, preferably dextran T-40, or polyvinylpyrrolidone (PVP), preferably in the range of 30,000-40,000 MW, may also be used at concentrations at about 5% w/v or less, preferably between about 0.01% w/v to about 0.5% w/v, more preferably between about 0.025% w/v to about 0.2% w/v, most preferably about 0.05% w/v. Other cell culture grade and cell-compatible agents that enhance collagen processing and deposition may be ascertained by the skilled routineer in the art of mammalian cell culture.

When the cell producing cells are confluent, and the culture medium is supplemented with components that assist in matrix synthesis, secretion, or organization, the cells are said to be stimulated to form a tissue-construct comprised of cells and matrix synthesized by those cells.

Therefore, a preferred matrix production medium formulation comprises: a base comprising Dulbecco's Modified Eagle's Medium (DMEM) (high glucose formulation, without L-glutamine) supplemented with either 4 mM L-glutamine or equivalent, 5 ng/ml epidermal growth factor, 0.4 μg/ml hydrocortisone, 1×10⁻⁴ M ethanolamine, 1×10⁻⁴ M o-phosphoryl-ethanolamine, 5 μg/ml insulin, 5 μg/ml transferrin, 20 ρM triiodothyronine, 6.78 ng/ml selenium, 50 ng/ml L-ascorbic acid, 0.2 μg/ml L-proline, and 0.1 μg/ml glycine. To the production medium, other pharmacological agents may be added to the culture to alter the nature, amount, or type of the extracellular matrix secreted. These agents may include polypeptide growth factors, transcription factors or inorganic salts to up-regulate collagen transcription. Examples of polypeptide growth factors include transforming growth factor-beta 1 (TGF-β1) and tissue-plasminogen activator (TPA), both of which are known to upregulate collagen synthesis. Raghow et al., Journal of Clinical Investigation, 79:1285-1288 (1987); Pardes et al., Journal of Investigative Dermatology, 100:549 (1993). An example of an inorganic salt that stimulates collagen production is cerium. Shivakumar et al., Journal of Molecular and Cellular Cardiology 24:775-780 (1992).

### II. Cell Types

An extracellular matrix-producing cell type for use in the invention may be any cell type capable of producing and secreting extracellular matrix components and organizing the extracellular matrix components to form a cell-matrix construct. More than one extracellular matrix-producing cell type may be cultured to form a cell-matrix construct. Cells of different cell types or tissue origins may be cultured together as a mixture to produce complementary components and structures similar to those found in native tissues. For example, the extracellular matrix-producing cell type may have other cell types mixed with it to produce an amount of extracellular matrix that is not normally produced by the first cell type. Alternatively, the extracellular matrix-producing cell type may also be mixed with other cell types that form specialized tissue structures in the tissue but do not substantially contribute to the overall formation of the matrix aspect of the cell-matrix construct, such as in certain skin constructs of the invention.

While any extracellular matrix-producing cell type may be used in accordance with this invention, the preferred cell types for use in this invention are derived from mesenchyme. More preferred cell types are fibroblasts, stromal cells, and other supporting connective tissue cells, most preferably human dermal fibroblasts found in human dermis for the production of a human dermal construct. Fibroblast cells, generally, produce a number of extracellular matrix proteins, primarily collagen. There are several types of collagens produced by fibroblasts, however, type I collagen is the most prevalent in vivo. Human fibroblast cell strains can be derived from a number of sources, including, but not limited to neonate male foreskin, dermis, tendon, lung, umbilical cords, cartilage, urethra, corneal stroma, oral mucosa, and intestine. The human cells may include but need not be limited to fibroblasts, but may include: smooth muscle cells, chondrocytes and other connective tissue cells of mesenchymal origin. It is preferred, but not required, that the origin of the matrix-producing cell used in the production of a tissue construct be derived from a tissue type that it is to resemble or mimic after employing the culturing methods of the invention. While not wishing to be bound by theory, dermal fibroblasts such as those derived from neonatal fibroblasts have wide application for most tissues in the body. Benefits of neonatal dermal fibroblasts is that they are believed to have plastic qualities, meaning that they are capable of transdifferentiation; are ideal for an hypoxic environment; and, are believed to be safe, biocompatible, and immuno-privileged as to not induce rejection by the subject. In another preferred embodiment, fibroblasts isolated by microdissection from the dermal papilla of hair follicles can be used to produce the matrix alone or in association with other fibroblasts. In the embodiment where a corneal-construct is produced, the matrix-producing cell is derived from corneal stroma. Cell donors may vary in development and age. Cells may be derived from donor tissues of embryos, neonates, or older individuals including adults. Embryonic progenitor cells such as mesenchymal stem cells may be used in the invention and induced to differentiate to develop into the desired tissue.

Although human cells are preferred for use in the invention, the cells to be used in the method of the invention are not limited to cells from human sources. Cells from other mammalian species including, but not limited to, equine, canine, porcine, bovine, and ovine sources; or rodent species such as mouse or rat may be used. In addition, cells that are spontaneously, chemically or virally transfected or recombinant cells or genetically engineered cells may also be used in this invention. For those embodiments that incorporate more than one cell type, chimeric mixtures of normal cells from two or more sources, such as a chimeric mixture of autologous and allogeneic cells; mixtures of normal and genetically modified or transfected cells; mixtures of cells derived from different tissue or organ types; or, mixtures of cells of two or more species or tissue sources may be used.

Recombinant or genetically-engineered cells may be used in the production of the cell-matrix construct to create a tissue construct that acts as a drug delivery graft for a subject needing increased levels of natural cell products or treatment with a therapeutic. The cells may produce and deliver to the subject via the graft recombinant cell products, growth factors, hormones, peptides or proteins for a continuous amount of time or as needed when biologically, chemically, or thermally signaled due to the conditions present in the subject. Either long or short-term gene product expression is desirable, depending on the use indication of the cultured tissue construct. Long term expression is desirable when the cultured tissue construct is implanted to deliver therapeutic products to a subject for an extended period of time. Conversely, short term expression is desired in instances where the cultured tissue construct is grafted to a subject having a wound where the cells of the cultured tissue construct are to promote normal or near-normal healing or to reduce scarification of the wound site. Once the wound has healed, the gene products from the cultured tissue construct are no longer needed or may no longer be desired at the site. Cells may also be genetically engineered to express proteins or different types of extracellular matrix components which are either ‘normal’ but expressed at high levels or modified in some way to make a graft device comprising extracellular matrix and living cells that is therapeutically advantageous for improved wound healing, facilitated or directed neovascularization, or minimized scar or keloid formation. These procedures are generally known in the art, and are described in Sambrook et al, Molecular Cloning A Laboratory Manual, Cold Spring Harbor Press, Cold Spring Harbor, N.Y. (1989), incorporated herein by reference. All of the above-mentioned types of cells are included within the definition of a “matrix-producing cell” as used in this invention.

The predominant major extracellular matrix component produced by fibroblasts is fibrillar collagen, particularly collagen type I. Fibrillar collagen is a key component in the cell-matrix structure; however, this invention is not to be limited to matrices comprised of only this protein or protein type. For instance, other collagens, both fibrillar and non-fibrillar collagen from the collagen family such as collagen types TI, ITT, IV, V, VI, VII, VIII, IX, X, XI, XII, XIII, XIV, XV, XVI, XVII, XVIII, XIX, and others as they may be identified may be produced by use of the appropriate cell type. Similarly, other matrix proteins which can be produced and deposited using the current method include, but are not limited to elastin; proteoglycans such as decorin or biglycan; or glycoproteins such as tenascin; vitronectin; fibronectin; laminin, thrombospondin I, and glycosaminoglycans (GAG) such as hyaluronic acid (HA).

As the aforementioned cell types may be used to produce the cell-matrix of the invention, they may also be delivered by the cell-matrix compositions of the invention where one or more cell-matrix sheets, in living, devitalized, or decellularized form are fabricated into a cell delivery device. These cell types may be delivered in contact with the cell-matrix of the invention to a site in a subject needing functional cells or cell products. As the cell-matrix compositions of the invention comprise collagen and collagen is a natural substrate for cell adhesion, these cells will naturally adhere to the cell-matrix composition. As the cell-matrix composition is also handleable, it allows for delivery of the cells and acts as a means for keeping the cells local to the delivery site.

### III. Culture Conditions and Methods

The system for the production of the cell-matrix layer may be either static or may employ a perfusion means to the culture media to exert a mechanical force against the forming cell-matrix layer to mimic in vivo forces. The application of appropriate stimuli may result in desirable properties, e.g., increased strength, as compared to static cultures. In the static system, the culture medium is still and relatively motionless as contrasted to the perfusion system where the medium is in motion. The perfusion of medium affects the viability of the cells and augments the development of the matrix layer. Perfusion means include, but are not limited to: using a magnetic stirbar or motorized impeller in the culture dish subjacent (below) or adjacent to the substrate carrier containing the culture membrane to stir the medium; pumping medium within or through the culture dish or chamber; gently agitating the culture dish on a shaking or rotating platform; or rolling, if produced in a roller bottle. Other perfusion means can be determined by one skilled in the art for use in the method of the invention. Other mechanical forces may be exerted by pulsing, flexing, undulating or stretching of the porous membrane during culture.

The cultures are maintained in an incubator to ensure sufficient environmental conditions of controlled temperature, humidity, and gas mixture for the culture of cells. Preferred conditions are between about 34° C. to about 38° C., more preferably 37±1° C. with an atmosphere between about 5-10±1% CO₂ and a relative humidity (Rh) between about 80-90%. One of skill in the art may easily determine environmental conditions that may be inside our outside the aforementioned environmental conditions depending on the cells being cultured or the step of culture being performed. Cultures may be removed from these conditions to ambient room temperature, air, and humidity such as during feeding, the seeding of additional cells, or other treatment without detriment to the cultures or their ability to form a cell-matrix sheet.

In the preferred embodiment, the cell-matrix construct is a dermal construct formed of dermal fibroblasts and their secreted matrix. Preferably, human dermal fibroblasts are used, derived as primary cells from dermis or more preferably from serially passaged or subcultured from established cell stocks or banks that have been screened against viral and bacterial contamination and tested for purity. Cells are cultured under sufficient conditions in growth medium to cause them to proliferate to an appropriate number for seeding the cells to the culture substrate on which to form a cell-matrix construct. Alternatively, cells from frozen cell stocks may be seeded directly to the culture substrate.

Once sufficient cell numbers have been obtained, cells are harvested and seeded onto a suitable culture surface and cultured under appropriate growth conditions to form a confluent sheet of cells. In the preferred embodiment, the cells are seeded on a porous membrane that is submerged to allow medium contact from both below the cell culture through the pores and above with contact above on the top surface of the cell culture. Preferably, cells are suspended in either base or growth media and are seeded on the cell culture surface at a density between about 1×10⁵ cells/cm² to about 6.6×10⁵ cells/cm², more preferably between about 3×10⁵ cells/cm² to about 6.6×10⁵ cells/cm²′ and most preferably at about 6.6×10⁵ cells/cm² (cells per square centimeter area of the surface). Cultures are cultured in growth medium to establish the culture and are cultured to between about 80% to 100% confluence at which time they are induced chemically by changing the medium to matrix production medium in order to upregulate the synthesis and secretion of extracellular matrix. In an alternate method, cells are seeded directly in matrix production medium at least 80% confluence to eliminate the need to change from the basic medium to the production medium but it is a method that requires higher seeding densities. Higher seeding densities achieve a level of super-confluence, meaning that the cells are seeded at over 100% confluence up to about 900% confluence, including in the range of about 300% to about 600% confluence. When seeded at super-confluence, the growth phase of culturing cells on the membrane is bypassed and the cells are seeded in the matrix production medium in order to start matrix production at the time of seeding.

During the culture, fibroblasts secrete endogenous matrix molecules and organize the secreted matrix molecules to form a three dimensional tissue-like structure but do not exhibit significant contractile forces to cause the forming cell-matrix construct to contract and peel itself from the culture substrate. Media exchanges are made every two to three days with fresh matrix production medium and with time, the secreted matrix increases in thickness and organization. The time necessary for creating a cell-matrix construct is dependent on the initial seeding density, the cell type, the age of the cell line, and the ability of the cell line to synthesize and secrete matrix components. When fully formed, the cell-matrix constructs of the invention have bulk thickness due to the fibrous matrix produced and organized by the cells; they are not ordinarily confluent or overly confluent cell cultures where the cells may be loosely adherent to each other. The fibrous quality gives the constructs cohesive tissue-like properties unlike ordinary cultures because they resist physical damage, such as tearing or cracking, with routine handling in a clinical setting. In the fabrication of a cultured cell-matrix sheet from dermal fibroblasts to form a dermal construct, the cells will form an organized matrix around themselves on the cell culture surface preferably at least about 30 microns in thickness or more, more preferably between about 60 to about 120 microns thick across the surface of the membrane; however, thicknesses have been obtained in excess of 120 microns and are suitable for use in testing or clinical applications where such greater thicknesses are needed.

### VI. Culture Substrate

The matrix-producing cell is cultured in a vessel suitable for animal cell or tissue culture, such as a culture dish, flask, or roller-bottle, which allows for the formation of a three-dimensional tissue-like structure. Suitable cell growth surfaces on which the cells can be grown can be any biologically compatible material to which the cells can adhere and provide an anchoring means for the cell-matrix construct to form. Materials such as glass; stainless steel; polymers, including polycarbonate, polystyrene, polyvinyl chloride, polyvinylidene, polydimethylsiloxane, fluoropolymers, and fluorinated ethylene propylene; and silicon substrates, including fused silica, polysilicon, or silicon crystals may be used as a cell growth surfaces. The cell growth surface material may be chemically treated or modified, electrostatically charged, or coated with biologicals such as poly-1-lysine or peptides. An example of a peptide coating is RGD peptide.

While the tissue construct of the invention may be grown on a solid cell growth surface, a cell growth surface with pores that communicate both top and bottom surfaces of the membrane to allow bilateral contact of the medium to the developing tissue construct or for contact from only below the culture is preferred. Bilateral contact allows medium to contact both the top and bottom surfaces of the developing cell-matrix-based construct for maximal surface area exposure to the nutrients contained in the medium. Medium may also contact only the bottom of the forming cultured tissue construct so that the top surface may be exposed to air, as in the development of a cultured skin construct. The preferred culture vessel is one that utilizes a carrier or culture insert, a culture-treated permeable member such as a porous membrane that is suspended in the culture vessel containing medium. Typically, the membrane is secured to one end of a tubular member or framework that is inserted within and interfaces with a base, such as a petri or culture dish that can be covered with a lid. Culture vessels incorporating a carrier insert with a porous membrane are known in the art and are preferred for carrying out the invention and are described in a number United States patents in the field, some of which have been made commercially available, including for instance: U.S. Pat. Nos. 5,766,937, 5,466,602, 5,366,893, 5,358,871, 5,215,920, 5,026,649, 4,871,674, 4,608,342, the disclosures of which are incorporated herein. When these types of culture vessels are employed, the tissue-construct is produced on one surface of the membrane, preferably the top, upwardly facing surface and the culture is contacted by cell media on both top and bottom surfaces. The pores in the growth surface allow for the passage of culture media for providing nutrients to the underside of the culture through the membrane, thus allowing the cells to be fed bilaterally or solely from the bottom side. A preferred pore size is one that is small enough that it does not allow for the growth of cells through the membrane, yet large enough to allow for free passage of nutrients contained in culture medium to the bottom surface of the cell-matrix construct, such as by capillary action. Preferred pore sizes are about less than 3 microns but range between about 0.1 microns to about 3 microns, more preferably between about 0.2 microns to about 1 micron and most preferably about 0.4 micron to about 0.6 micron sized pores are employed. In the case of human dermal fibroblasts, the most preferred material is polycarbonate having a pore size is between about 0.4 to about 0.6 microns. The maximum pore size depends not only on the size of the cell but also the ability of the cell to alter its shape and pass through the membrane. It is important that the tissue-like construct adheres to the surface but does not incorporate or envelop the substrate so it is removable from it such as by peeling with minimal force. The size and shape of the tissue construct formed is dictated by the size of the vessel surface or membrane on which it grown. Substrates may be round, square, rectangular or angular or shaped with rounded corner angles, or irregularly shaped. Substrates may also be flat or contoured as a mold to produce a shaped construct to interface with a wound or mimic the physical structure of native tissue. To account for greater surface areas of the growth substrate, proportionally more cells are seeded to the surface and a greater volume of media is needed to sufficiently bathe and nourish the cells. When the cell-matrix-based tissue construct is finally formed, it is removed by peeling from the membrane substrate before grafting to a subject.

The cultured cell-matrix constructs of the invention do not rely on synthetic or bioresorbable members for, such as a mesh member for formation. A mesh member is organized as a woven, a knit, or a felt material. In systems where a mesh member is employed, the cells are cultured on the mesh member and grow on either side and within the interstices of the mesh to envelop and incorporate the mesh within the cultured tissue construct. The final construct formed by methods that incorporate such a mesh rely on it for physical support and for bulk. Examples of cultures tissue constructs that rely on synthetic mesh members are found in U.S. Pat. Nos. 5,580,781, 5,443,950, 5,266,480, 5,032,508, 4,963,489 to Naughton, et al.

### IV. Chemical Modifications

The cell-matrix constructs of the invention are either devitalized, to terminate the cells, or decellularized to remove the cells, depending upon their ultimate use in treating a subject.

The cell-matrix of the invention may be devitalized or decellularized either on the membrane of the culture insert or it may first be removed from it. As the culture insert suspends the cell-matrix in the dish to allow bilateral contact with culture medium, the bilateral contact maybe leveraged when the cell-matrix is treated using a chemical devitalizing or decellularizing agent or when the cell-matrix construct is dried using air, light or irradiation. The culture insert is conveniently removable from the culture apparatus so it may be transferred to a different vessel where it may be subjected to or contacted with a devitalizing or decellularizing agent.

To devitalize cells in a cell-matrix means to terminate, but not remove, the cells, to form a non-living cell-matrix. The constructs of the invention may be devitalized, in other words, the matrix-producing cells that produce the endogenous extracellular matrix components to form the cell-matrix constructs are terminated. When the cells are terminated, they remain in the matrix they formed. Devitalizing agents and methods are preferably those that retain the cell-matrix integrity and structure.

One method for devitalizing the cells in the cell-matrix construct employs dehydrating or drying the construct to remove all or substantially call of the moisture in the construct. Means for removing moisture include dehydration in air, by freezing or by freeze-drying. To dehydrate the construct by air-drying, culture medium is removed from the vessel in which the cell-matrix construct is made and the cell-matrix construct is simply allowed to dehydrate for a sufficient time to allow the cells to die. Dehydration conditions vary in terms of temperature and relative humidity. Preferred dehydration temperatures range from above freezing temperature up to the denaturation temperature of the collagen (as measured by differential scanning calorimetry, or “DSC”) in the cell-matrix construct, for example, between about 0° C. to about 60° C. A more preferred dehydration temperature is ambient room temperature, about 18° C. to about 22° C. Relative humidity values that are lower, as in the range of about 0% to about 60%, are preferred; however, relative humidities comparative to room humidity, between about 10% Rh to about 40% Rh are also preferred. When dehydration is conducted by air-drying at ambient room temperature and humidity, the cell-matrix construct will have about 10% to about 40% w/w moisture, or less. Therefore, when air-drying the cell-matrix constructs of the invention, some level of moisture is retained. To freeze-dry the construct, also termed “lyophilization”, the cell-matrix is frozen and then placed in a vacuum environment to remove the moisture. Lyophilization techniques can be employed to the constructs disclosed in the present invention such that biological activity of multiple growth factors within the constructs remain uninterrupted. In one aspect, one-layer cell-matrix constructs can be taken straight out of culture and frozen at −80° C., and lyophilized overnight, such as between about 6 to about 15 hours, or longer. In another aspect, one-layer cell-matrix constructs can first be air-dried for about eight hours, and subsequently frozen at −80° C., and lyophilized overnight, such as between about 6 to about 15 hours, or longer.

After drying in ambient conditions or by freeze-drying, the cell-matrix is devitalized but still retains devitalized cells and cell remnants. Lyophilization can also impart qualities different than those that may result when dehydrating under ambient conditions. Such qualities, in one embodiment, exhibits a more porous and open fibrous matrix structure.

Chemical means may also be employed to devitalize the cells in the cell-matrix construct. Water to osmotically terminate the cells may be used. Cell-matrix constructs are immersed in sterile, pure water for a time sufficient to allow for hypotonic swelling to cause the cells to lyse. After the cells lyse, the cell-matrix is devitalized but still retains devitalized cells and cell remnants. When water is used, it may also be mixed with other substances such as peracetic acid or hydrogen peroxide, or salts, or a combination thereof. For example, a devitalizing solution of peracetic acid between about 0.05% to about 3% v/v in water may be used. This devitalizing agent may also be buffered or contain a high salt concentration to prevent excessive swelling of the cell-matrix when terminating the cells.

Organic solvents and organic solvent solutions may be used as devitalizing agents in the invention. Organic solvents are capable of displacing the water in a cell-matrix construct to terminate, therefore, devitalize the cells in the cell-matrix. Preferably, the organic solvent employed to remove water is one that leaves no residues when they it is removed from the construct. Preferred organic solvents include alcohols, such as ethyl alcohol, methyl alcohol and isopropyl alcohol; or acetone. For the purpose of illustration, cell-matrix constructs are immersed in sterile ethyl alcohol for a time sufficient to displace water in the cell-matrix construct and devitalize the cells. The cell-matrix constructs are then removed from the ethyl alcohol and then exposed to air for a time sufficient to allow the absorbed ethyl alcohol in the cell-matrix construct to evaporate. After evaporation of solvent, the cell-matrix is devitalized but still retains the devitalized cells and cell remnants and the cell-matrix is dehydrated.

Other means to devitalize the cells include subjecting the cell-matrix constructs to ultraviolet light or gamma irradiation. These means may be used in conjunction with hypotonic swelling of the cell-matrix construct with water, or other chemical devitalizing means or with air and freezing devitalizing means.

To decellularize a cell-matrix of the invention means to remove the cells from the cell-matrix such that cells, cell remnants are removed from the cell-matrix to result in a extracellular matrix without the cells that produced it. The cell-matrix constructs of the invention may be decellularized, in other words, the matrix-producing cells that produce the endogenous extracellular matrix components to form a the cell-matrix constructs are removed from the cell-matrix. When the cells are removed, a cell-matrix endogenously produced by cultured cells now remains but without those cells that formed it. One preferred method for decellularizing the cell-matrix constructs of the invention uses a series of chemical treatments to remove the cells, cell remnants, and residual cellular DNA and RNA. Other non-collagenous and non-elastinous extracellular matrix components may also be removed or reduced with the agents and methods used to decellularized the cell-matrix constructs, such as glycoproteins, glycosaminoglycans, proteoglycans, lipids, and other non-collagenous proteins. The removal of cells and non-collagenous and non-elastinous components from the cell-matrix yields a cell-matrix that is acellular and comprised of all or substantially all collagen with some lesser amounts of elastin.

The cell-matrix construct is first treated by contacting it with an effective amount of chelating agent, preferably physiologically alkaline to controllably limit swelling of the cell-matrix. Chelating agents enhance removal of cells, cell debris and basement membrane structures from the matrix by reducing divalent cation concentration. Alkaline treatment dissociates glycoproteins and glycosaminoglycans from the collagenous tissue and saponifies lipids. Chelating agents known in the art which may be used include, but are not limited to, ethylenediaminetetraacetic acid (EDTA) and ethylenebis(oxyethylenitrilo)tetraacetic acid (EGTA). EDTA is a preferred chelating agent and may be made more alkaline by the addition of sodium hydroxide (NaOH), calcium hydroxide Ca(OH)₂, sodium carbonate or sodium peroxide. EDTA or EGTA concentration is preferably between about 1 to about 200 mM; more preferably between about 50 to about 150 mM; most preferably around about 100 mM. NaOH concentration is preferably between about 0.001 to about 1 M; more preferably between about 0.001 to about 0.10 M; most preferably about 0.01 M. Other alkaline or basic agents can be determined by one of skill in the art to bring the pH of the chelating solution within the effective basic pH range. The final pH of the basic chelating solution should be preferably between about 8 and about 12, but more preferably between about 11.1 to about 11.8. In the most preferred embodiment, the cell-matrix is contacted with a solution of 100 mM EDTA/10 mM NaOH in water. The cell-matrix is contacted preferably by immersion in the alkaline chelating agent while more effective treatment is obtained by gentle agitation of the construct and the solution together for a time for the treatment step to be effective.

The cell-matrix is then contacted with an effective amount of acidic solution, preferably containing a salt. Acid treatment also plays a role in the removal of glycoproteins and glycosaminoglycans as well as in the removal of non-collagenous proteins and nucleic acids such as DNA and RNA. Salt treatment controls swelling of the collagenous matrix during acid treatment and is involved with removal of some glycoproteins and proteoglycans from the collagenous matrix. Acid solutions known in the art may be used and may include but are not limited to hydrochloric acid (HCl), acetic acid (CH₃COOH) and sulfuric acid (H₂SO₄). A preferred acid is hydrochloric acid (HCl) at a concentration preferably between about 0.5 to about 2 M, more preferably between about 0.75 to about 1.25 M; most preferably around 1 M. The final pH of the acid/salt solution is preferably between about 0 to about 1, more preferably between about 0 and 0.75, and most preferably between about 0.1 to about 0.5. Hydrochloric acid and other strong acids are most effective for breaking up nucleic acid molecules while weaker acids are less effective. Salts that may be used are preferably inorganic salts and include but are not limited to chloride salts such as sodium chloride (NaCl), calcium chloride (CaCl₂), and potassium chloride (KCl) while other effective salts may be determined by one of skill in the art. Preferably chloride salts are used at a concentration preferably between about 0.1 to about 2 M; more preferably between about 0.75 to about 1.25 M; most preferably around 1 M. A preferred chloride salt for use in the method is sodium chloride (NaCl). In the most preferred embodiment, the cell-matrix is contacted with 1 M HCl/1 M NaCl in water. The cell-matrix is contacted preferably by immersion in the acid/salt solution while effective treatment is obtained by gentle agitation of the construct and the solution together for a time for the treatment step to be effective.

The cell-matrix is then contacted with an effective amount of salt solution which is preferably buffered to about a physiological pH. The buffered salt solution neutralizes the material while reducing swelling. Salts that may be used are preferably inorganic salts and include but are not limited to chloride salts such as sodium chloride (NaCl), calcium chloride (CaCl₂), and potassium chloride (KCl); and nitrogenous salts such as ammonium sulfate (NH₃SO₄) while other effective salts may be determined by one of skill in the art. Preferably chloride salts are used at a concentration preferably between about 0.1 to about 2 M; more preferably between about 0.75 to about 1.25 M; most preferably about 1 M. A preferred chloride salt for use in the method is sodium chloride (NaCl). Buffering agents are known in the art and include but are not limited to phosphate and borate solutions while others can be determined by the skilled artisan for use in the method. One preferred method to buffer the salt solution is to add phosphate buffered saline (PBS) preferably wherein the phosphate is at a concentration from about 0.001 to about 0.02 M and a salt concentration from about 0.07 to about 0.3 M to the salt solution. A preferred pH for the solution is between about 5 to about 9, more preferably between about 7 to about 8, most preferably between about 7.4 to about 7.6. In the most preferred embodiment, the tissue is contacted with 1 M sodium chloride (NaCl)/10 mM phosphate buffered saline (PBS) at a pH of between about 7.0 to about 7.6. The cell-matrix is contacted preferably by immersion in the buffered salt solution while effective treatment is obtained by gentle agitation of the tissue and the solution together for a time for the treatment step to be effective.

After chemical cleaning treatment, the cell-matrix is then preferably rinsed free of chemical cleaning agents by contacting it with an effective amount of rinse agent. Agents such as water, isotonic saline solutions and physiological pH buffered solutions can be used and are contacted with the cell-matrix for a time sufficient to remove the cleaning agents. A preferred rinse solution is physiological pH buffered saline such as phosphate buffered saline (PBS). Other means for rinsing the cell-matrix of chemical cleaning agents can be determined by one of skill in the art. The cleaning steps of contacting the cell-matrix with an alkaline chelating agent and contacting the cell-matrix with an acid solution containing salt may be performed in either order to achieve substantially the same cleaning effect. The solutions may not be combined and performed as a single step, however.

The result of decellularizing a cell-matrix construct is an endogenously produced collagenous matrix produced by cultured cells that has been decellularized of the cells that produced it. A further result of decellularized cell-matrix construct is an endogenously produced collagenous matrix produced by cultured cells that has been decellularized of the cells that produced it and has a removal or reduction of non-collagenous and non-elastinous extracellular matrix components.

In some embodiments, the cell-matrix constructs may be first devitalized to terminate the cells and then decellularized to remove the devitalized cells.

The devitalized or decellularized cell-matrix constructs may be used in a current state but they may be further modified with chemical treatments, physical treatments, the addition of other substances such as drugs, growth factors, cultured cells, other matrix components of natural, biosynthetic, polymeric origin, and they may be combined with medical devices such as stents and closure devices for treating patent foramen ovale defects in the heart.

Crosslinking. The decellularized or devitalized cell-matrix may be crosslinked using a crosslinking agent to control its rate of bioremodeling and to either increase its persistence when implanted or engrafted into a living body. It may be crosslinked and used as a single layer construct or it may be combined or manipulated to create different types of constructs. The crosslinking methods of the invention also provide for methods of bonding cell-matrix sheets, or portions thereof, together.

The cell-matrix is preferably a planar sheet structure that can be used to fabricate various types of cell-matrix constructs to be used as a prosthesis with the shape of the prosthesis ultimately depending on its intended use. To form prostheses of the invention, the devitalized or decellularized cell-matrix sheets should be fabricated using a method that preserves the bioremodelability of the matrix sheets but also is able to enhance its strength and structural characteristics for its performance as a replacement tissue. Flat-sheet constructs of the invention comprise either devitalized or decellularized cell-matrix sheets, or devitalized and decellularized matrix sheets (such as one devitalized cell-matrix sheet and one decellularized cell-matrix sheet) layered to contact another, and bonded together. Tubular constructs of the invention comprise either a devitalized or decellularized matrix sheet rolled over itself to at least a minimum degree to contact itself. The area of contact between matrix sheets or a matrix sheet to itself is a bonding region.

Multilayer crosslinked constructs. In a preferred embodiment, the prosthetic device of this invention has two or more superimposed matrix sheets that are bonded together to form a flat-sheet construct. As used herein, “bonded collagen layers” means composed of two or more cell-matrix sheets of the same or different origins or profiles treated in a manner such that the layers are superimposed on each other and are sufficiently held together by self-lamination and chemical bonding.

A preferred embodiment of the invention is directed to flat sheet prostheses, and methods for making and using flat sheet prostheses, comprising of two or more matrix sheets that are bonded and crosslinked. Due to the flat sheet structure of the matrix sheets, the prosthesis is easily fabricated to comprise any number of layers, preferably between 2 and 20 layers, more preferably between 2 and 10 layers, with the number of layers depending on the strength and bulk necessary for the final intended use of the construct. Alternatively, as the ultimate size of a superimposed arrangement is limited by the size of the matrix sheets, the layers may be staggered, in a collage arrangement to form a sheet construct with a surface area larger than the dimensions of any individual matrix sheet but without continuous layers across the area of the arrangement.

In the fabrication of a multilayer construct comprising matrix sheets, an aseptic environment and sterile tools are preferably employed to maintain sterility. To form a multilayer construct of matrix sheets, a first sterile rigid support member, such as a rigid sheet of polycarbonate, is laid down. If the matrix sheets are still not in a hydrated state from the devitalizing or decellularizing processes, they are hydrated in aqueous solution, such as water or phosphate buffered saline. Matrix sheets are blotted with sterile absorbent cloths to absorb excess water from the material. A first matrix sheet is laid on the polycarbonate sheet and is manually smoothed to the polycarbonate sheet to remove any air bubbles, folds, and creases. A second matrix sheet is laid on the top of the first sheet, again manually removing any air bubbles, folds, and creases. This layering is repeated until the desired number of layers for a specific application is obtained.

After layering the desired number of matrix sheets, they are then dehydrated together. While not wishing to be bound by theory, dehydration brings the extracellular matrix components, such as collagen fibers, in the layers together when water is removed from between the fibers of the adjacent matrix sheets. The layers may be dehydrated either open-faced on the first support member or, between the first support member and a second support member, such as a second sheet of polycarbonate, placed before drying over the top layer and fastened to the first support member to keep all the layers in flat planar arrangement together with or without compression. To facilitate dehydration, the support member may be porous to allow air and moisture to pass through to the dehydrating layers. The layers may be dried in air, in a vacuum, or by chemical means such as by acetone or an alcohol such as ethyl alcohol or isopropyl alcohol. Dehydration by air-drying may be done to room humidity, between about 0% Rh to about 60% Rh, or less; or about 10% to about 40% w/w moisture, or less. Dehydration may be easily performed by angling the superimposed matrix layers to face a sterile airflow of a laminar flow cabinet for at least about 1 hour up to 24 hours at ambient room temperature, approximately 20° C., and at room humidity. Dehydration conducted by vacuum or chemical means will dehydrate the layers to moisture levels lower than those achieved by air-drying.

In an optional step, the dehydrated layers are rehydrated or, alternatively, rehydrated and dehydrated again. As mentioned above, the dehydration brings the extracellular matrix components of adjacent matrix layers together and crosslinking those layers together forms chemical bonds between the components to bond the layers. To rehydrate the layers, they are peeled off the porous support member together and are rehydrated in an aqueous rehydration agent, preferably water, by transferring them to a container containing aqueous rehydration agent for at least about 10 to about 15 minutes at a temperature between about 4° C. to about 20° C. to rehydrate the layers without separating or delaminating them. The matrix layers are then crosslinked together by contacting the layered matrix sheets with a crosslinking agent, preferably a chemical crosslinking agent that preserves the bioremodelability of the matrix layers.

Crosslinking the bonded prosthetic device also provides strength and durability to the device to improve handling properties. Various types of crosslinking agents are known in the art and can be used such as carbodiimides, genipin, transglutaminase, ribose and other sugars, nordihydroguaiaretic acid (NDGA), oxidative agents, ultraviolet (UV) light and dehydrothermal (DHT) methods. Besides chemical crosslinking agents, the layers may be bonded together with biocompatible fibrin-based glues or medical grade adhesives such as polyurethane, vinyl acetate or polyepoxy. One preferred biocompatible adhesive is silk fibroin, that is a 4-8% silk fibroin solution disposed at the bonding region between adjacent layers of tissue matrix that is activated using methyl alcohol. Biocompatible glues or adhesives may be used to bond crosslinked or uncrosslinked layers, or both, together to form bioengineered constructs of the invention.

A preferred biocompatible adhesive is silk fibroin, that is about a 2-8% silk fibroin solution disposed at the bonding region between adjacent layers of tissue matrix. In one aspect, two or more cell-matrix constructs described above can be combined using biocompatible adhesive biomaterials. As an example, the silk fibroin solution can be obtained from Bombyx mori silkworm, which can be processed to obtain a sericin-free compound, which, in one aspect, can be used as a biocompatible, silk adhesive. Bombyx mori consists primarily of glycine and alanine repeats that dominate the structure. The fibroin chain consists of two basic polypeptide sequences, crystalline and less ordered polypeptides that alternate regularly. The basic sequence of the ‘crystalline’ polypeptides is of -(Ala-Gly)ₙ- that adopts a β-sheet structure, whereas the ‘less ordered’ polypeptides contain additional amino acids, in particular, tyrosine, valine and acidic as well as basic amino acids. It is to be appreciated that a silk fibroin derived from recombinant source may be used to achieve a similar biocompatible adhesive properties to carry out the invention.

Briefly, in one aspect, cocoons of B. mori silkworm are boiled for 20 to 30 minutes in an aqueous solution comprising 0.02 M Na₂CO₃. In order to extract the glue-like sericin proteins, the cocoons are subsequently rinsed. In one embodiment, the extracted silk fibroin is dissolved in 9.3 M Lithium-Bromide (LiBr) solution at about 60° C. for about 4 hours, which yields a 20% weight by volume (w/v) solution. The resulting solution is subsequently dialyzed against distilled water using a Slide-a-Lyzer dialysis cassette (MWCO 3,500, Pierce) at room temperature for 48 h to remove the salt, however any dialyzing procedure is within the contemplation of the invention. The resulting dialysate is centrifuged in duplicate, each at −20° C. for 20 minutes in order to remove impurities and aggregates formed during the dialysis step.

A preferred crosslinking agent is 1-ethyl-3-(3-dimethylaminopropyl) carbodiimide hydrochloride (EDC). In an another preferred method, sulfo-N-hydroxysuccinimide is added to the EDC crosslinking agent as described by Staros, J. V., Biochem. 21, 3950-3955, 1982. In the most preferred method, EDC is solubilized in water at a concentration preferably between about 0.1 mM to about 100 mM, more preferably between about 1.0 mM to about 10 mM, most preferably at about 1.0 mM. Besides water, phosphate buffered saline or (2-[N-morpholino]ethanesulfonic acid) (MES) buffer may be used to dissolve the EDC. Other agents may be added to the solution, such as acetone or an alcohol, up to 99% v/v in water, typically 50%, to make crosslinking more uniform and efficient. These agents remove water from the layers to bring the matrix fibers together to promote crosslinking between those fibers. The ratio of these agents to water in the crosslinking agent can be used to regulate crosslinking. EDC crosslinking solution is prepared immediately before use as EDC will lose its activity over time. To contact the crosslinking agent to the matrix layers, the hydrated, bonded matrix layers are transferred to a container such as a shallow pan and the crosslinking agent gently decanted to the pan ensuring that the matrix layers are both covered and free-floating and that no air bubbles are present under or between the matrix layers. The container is covered and the matrix layers are allowed to crosslink for between about 4 to about 24 hours, more preferably between 8 to about 16 hours at a temperature between about 4° C. to about 20° C. Crosslinking can be regulated with temperature: At lower temperatures, crosslinking is more effective as the reaction is slowed; at higher temperatures, crosslinking is less effective as the EDC is less stable.

After crosslinking, the crosslinking agent is decanted and disposed of and the crosslinked multi-layer matrix constructs are rinsed by contacting them with a rinse agent to remove residual crosslinking agent. A preferred rinse agent is water or other aqueous solution. Preferably, sufficient rinsing is achieved by contacting the crosslinked multi-layer matrix constructs three times with equal volumes of sterile water for about five minutes for each rinse.

Tubular constructs. In another preferred embodiment, the matrix construct of this invention is a tubular construct formed from a single, generally rectangular matrix sheet. The matrix sheet is rolled so that one edge meets and overlaps an opposing edge. The overlap serves as a bonding region. The tubular construct formed from a matrix sheet may be fabricated in various diameters, lengths, and number of layers and may incorporate other components depending on the indication for its use.

To form a tubular construct, a mandrel is chosen with a diameter measurement that will determine the diameter of the formed construct. The mandrel is preferably cylindrical or oval in cross section and made of glass, stainless steel or of a nonreactive, medical grade composition. The mandrel may be straight, curved, angled, it may have branches or bifurcations, or a number of these qualities. The number of layers intended for the tubular construct to be formed corresponds with the number of times a matrix sheet is wrapped around a mandrel and over itself. The number of times the matrix sheet can be wrapped depends on the dimensions of the processed matrix sheet. For a two layer tubular construct, the width of the matrix sheet must be sufficient for wrapping the sheet around the mandrel at least twice. It is preferable that the width be sufficient to wrap the sheet around the mandrel the required number of times and an additional percentage more as an overlap, between about 5% to about 20% of the mandrel circumference, to secure the bonding region and to ensure a tight seam. Similarly, the length of the mandrel will dictate the length of the tube that can be formed on it. For ease in handling the construct on the mandrel, the mandrel should be longer than the length of the construct so the mandrel, and not the construct being formed, is contacted when handled.

It is preferred that the mandrel is provided with a covering of a nonreactive, medical grade quality, elastic, rubber or latex material in the form of a sleeve. While a tubular matrix sheet construct may be formed directly on the mandrel surface, the sleeve facilitates the removal of the formed tube from the mandrel and does not adhere to, react with, or leave residues on the matrix sheet. To remove the formed construct, the sleeve may be pulled from one end off the mandrel to carry the construct from the mandrel with it. Because the matrix sheet only lightly adheres to the sleeve and is more adherent to other matrix sheet, fabricating tubes from matrix sheets is facilitated as the tubulated contract may be removed from the mandrel without stretching or otherwise stressing or risking damage to the construct. In the most preferred embodiment, the sleeve comprises KRATON® (Shell Chemical Company), a thermoplastic rubber composed of styrene-ethylene/butylene-styrene copolymers with a very stable saturated midblock.

For simplicity in illustration, a two-layer tubular construct with a 4 mm diameter and a 10% overlap is formed on a mandrel having about a 4 mm diameter. The mandrel is provided with a KRATON® sleeve approximately as long as the length of the mandrel and longer than the construct to be formed on it. A matrix sheet is trimmed so that the width dimension is about 28 mm and the length dimension may vary depending on the desired length of the construct. In the sterile field of a laminar flow cabinet, the matrix sheet is then formed into a tube by the following process. The matrix sheet is moistened along one edge and is aligned with the sleeve-covered mandrel and, leveraging the adhesive nature of the matrix sheet, it is “flagged” along the length of the sleeve-covered mandrel and dried in position for at least 10 minutes or more. The flagged matrix sheet is then hydrated and wrapped around the mandrel and then over itself one full revolution plus 10% of the circumference, for a 110% overlap, to serve as a bonding region and to provide a tight seam.

For the formation of single layer tubular construct, the matrix sheet must be able to wrap around the mandrel one full revolution and at least about a 5% of an additional revolution as an overlap to provide a bonding region that is equal to about 5% of the circumference of the construct. For a two-layer construct, the matrix sheet must be able to wrap around the mandrel at least twice and preferably an additional 5% to 20% revolution as an overlap. While the two-layer wrap provides a bonding region of 100% between the matrix sheet surfaces, the additional percentage for overlap ensures a tight, impermeable seam. For a three-layer construct, the matrix sheet must be able to wrap around the mandrel at least three times. The construct may be prepared with any number of layers as limited by the dimensions of the matrix sheet and the specifications desired. Typically, a tubular construct will have 10 layers or less, such as between 2 to 6 layers or between 2 or 3 layers with varying degrees of overlap. After wrapping, any air bubbles, folds, and creases are smoothed out from under the material and between the layers.

Matrix sheets may be rolled onto the mandrel either manually or with the assistance of an apparatus that aids for even tensioning and smoothing out of air or water bubbles or creases that can occur under the mandrel or between the layers of the wrapped matrix sheet. The apparatus would have a surface that the mandrel can contact along its length as it is turned to wrap the matrix sheet.

The layers of the wrapped matrix sheet are then bonded together by employing the methods and agents used in bonding and crosslinking flat-sheet constructs made from matrix sheets. After crosslinking and rinsing, the wrapped dehydrated ICL constructs may be then pulled off the mandrel via the sleeve or left on for further processing. The constructs may be rehydrated in an aqueous solution, preferably water, by transferring them to a room temperature container containing rehydration agent for at least about 10 to about 15 minutes to rehydrate the layers without separating or delaminating them.

Addition of substances. The devitalized or decellularized single cell-matrix sheet or multi-layer cell-matrix construct of the invention may further comprise one or more additional substances to impart different handling or functional qualities or to impart different characteristics to the material so that cells and tissues in a living body will react in a desirable way to it when implanted or engrafted to, or in, the body.

Heparin. In embodiments where the construct will be used in contact with blood, as in the circulatory system, the construct is rendered non-thrombogenic by applying heparin to the construct, to all surfaces of the construct or one side only in a flat-sheet construct or either luminally or abluminally for a tubular construct. Heparin can be applied to the construct, by a variety of well-known techniques. For illustration, heparin can be applied to the construct in the following three ways. First, benzalkonium heparin (BA-Hep) isopropyl alcohol solution is applied to the prosthesis by vertically filling the lumen or dipping the prosthesis in the solution and then air-drying it. This procedure treats the collagen with an ionically bound BA-Hep complex. Second, EDC can be used to activate the heparin and then to covalently bond the heparin to the collagen fiber. Third, EDC can be used to activate the collagen, then covalently bond protamine to the collagen and then ionically bond heparin to the protamine.

Antimicrobial treatments, drugs, growth factors, cytokines, genetic material and cultured cells may be incorporated in or on the matrix layers. Additional material layers may be disposed on at least one surface of the constructs such additional material layers include proteins and other extracellular matrix components in purified or crude form.

Proteins. Extracellular matrix proteins are a preferred class of proteins for use in the present invention. Examples include but are not limited to collagen, fibrin, elastin, laminin, and fibronectin, proteoglycans. For example, the protein fibrinogen, when combined with thrombin, forms fibrin. Hyaluronan (also called hyaluronic acid or hyaluronate) is a non-sulfated glycosaminoglycan distributed widely throughout connective, epithelial, and neural tissues. It is one of the chief components of the extracellular matrix, contributes significantly to cell proliferation and migration and is used to reduce post-operative adhesions. There are multiple types of each of these proteins that are naturally-occurring as well as types that can be or are synthetically manufactured or produced by genetic engineering. For example, collagen occurs in many forms and types. All of these types and subsets are encompassed in the use of the proteins named herein. The term protein further includes, but is not limited to, fragments, analogs, conservative amino acid substitutions, and substitutions with non-naturally occurring amino acids with respect to each named protein. The term “residue” is used herein to refer to an amino acid (D or L) or an amino acid mimetic that is incorporated into a protein by an amide bond. As such, the amino acid may be a naturally occurring amino acid or, unless otherwise limited, may encompass known analogs of natural amino acids that function in a manner similar to the naturally occurring amino acids (i.e., amino acid mimetics). Moreover, an amide bond mimetic includes peptide backbone modifications well known to those skilled in the art.

Synthetic materials may be disposed upon on at least one surface of the cell-matrix constructs. The synthetic material may be in the form of a sheet, superimposed or staggered upon the cell-matrix construct to form a synthetic layer on the cell-matrix layer. One class of synthetic materials, preferably biologically compatible synthetic materials, comprises polymers. Such polymers include but are not limited to the following: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolid-es) (PLGA), polyanhydrides, and polyorthoesters or any other similar synthetic polymers that may be developed that are biologically compatible. The term “biologically compatible, synthetic polymers” shall also include copolymers and blends, and any other combinations of the forgoing either together or with other polymers generally. The use of these polymers will depend on given applications and specifications required. For example, biologically compatible synthetic materials may also be biodegradable such that, when implanted into the body of a subject, biodegrade over time. When disposed on a cell-matrix construct, the combination construct comprises a biodegradable layer and a bioremodelable layer. A more detailed discussion of these polymers and types of polymers is set forth in Brannon-Peppas, Lisa, “Polymers in Controlled Drug Delivery,” Medical Plastics and Biomaterials, November 1997, which is incorporated by reference as if set forth fully herein.

An example of another synthetic material that may be used as a backing layer is silicone. A silicone layer in the form of a porous or microporous membrane or a non-porous film is applied and adhered to a matrix construct. When used in wound healing, the silicone layer may be used to handle and maneuver the matrix construct to a skin wound and seal the wound periphery to enclose the matrix construct to treat the wound. The silicone also forms a moisture barrier to keep the wound from drying. Following successful formation of the healed wound tissue, typically at around 21 days, the silicone is peeled back carefully from the edges of the healed or healing wound with forceps.

Sterilization. Constructs are then terminally sterilized using means known in the art of medical device sterilization. A preferred method for sterilization is by contacting the constructs with sterile 0.1% peracetic acid (PA) treatment neutralized with a sufficient amount of 10 N sodium hydroxide (NaOH), according to U.S. Pat. No. 5,460,962, the disclosure of which is incorporated herein. Decontamination is performed in a container on a shaker platform, such as 1 L Nalge containers, for about 18.+−.2 hours. Constructs are then rinsed by contacting them with three volumes of sterile water for 10 minutes each rinse.

The constructs of the invention may be sterilized by gamma irradiation. Constructs are packaged in containers made from material suitable for gamma irradiation and sealed using a vacuum sealer, which were in turn placed in hermetic bags for gamma irradiation between 25.0 and 35.0 kGy. Gamma irradiation significantly, but not detrimentally, decreases Young's modulus and shrink temperature. The mechanical properties after gamma irradiation are still sufficient for use in a range of applications and gamma is a preferred means for sterilizing as it is widely used in the field of implantable medical devices.

### V. Physical Modifications

The construct of the present invention may also be meshed prior grafting to a subject in need of wound care. When used in wound healing, meshing improves conformation of the construct to the wound bed and provides a means for draining wound exudate from beneath the graft. The term ‘meshing’ is defined as a mechanical method by which a tissue is perforated with slits to form a net-like arrangement. Meshing is preferably obtained by the use of a conventional skin mesher (ZIMMER®; BIOPLASTY®). Meshed constructs may be expanded by stretching the skin so that the slits are opened and then applied to the wound bed. Expanded meshed constructs provides a wound area with maximal coverage. Alternatively, meshed constructs may be applied without expansion, simply as a sheet with an arrangement of unexpanded slits. The meshed construct may be applied alone or with the subject's own skin from another area of the body. Constructs of the invention may also have perforations or fenestrations and pores provided by other means. Fenestrations may be applied manually using a laser, punch, scalpel, needle or pin.

The construct of the present invention may also be provided holes that communicate between both planes of the construct. Holes are perforations that are introduced in a regular or irregular pattern. One could also manually score or perforate a tissue with a scalpel or a needle.

### VII. Treatment Methods

Bioengineered constructs of the invention may be used in wound healing, such as for acute wounds including surgical wounds or burn areas, or chronic wounds such as venous ulcers, diabetic ulcers, decubitus ulcers may experience a healing benefit by application of the disclosed skin construct. Other congenital skin diseases such as epidermolysis bullosa may benefit as well. Bioengineered constructs of the invention may be used in cardiac applications, periodontal applications, surgical applications, and cosmetic applications, and neurological applications, such as a dura mater repair patch or a graft for peripheral nerve repair, a wrap for nerve bundles or tube for guided nerve regeneration

Cell delivery. The bioengineered constructs of the invention may configured for, and used in cell delivery applications. Devitalized or decellularized constructs of the invention may be used as a culture substrate for cells. As the constructs of the invention primarily comprise collagen, they are a natural substrate for cell culture. A matrix construct of the invention may be placed and fixtured in a culture apparatus and a suspension of cells in culture medium disposed onto the matrix construct and allowed to attach and proliferate on the surface of the matrix construct, or within and below the surface of the matrix construct, or both. Chosen cells for culturing with the matrix construct are those that have qualities desirable for the treatment of a damaged or diseased organ or tissue to repair the organ or tissue to restore its intended functionality. For an example of a another configuration of a construct of the invention for cell delivery, the matrix layers may be configured as a pocket or envelope for delivery of stem or progenitor cells, precursor cells, or functional cells.

The following examples are provided to better explain the practice of the present invention and should not be interpreted in any way to limit the scope of the present invention. Those skilled in the art will recognize that various modifications can be made to the methods described herein while not departing from the spirit and scope of the present invention.

### EXAMPLES

### Example 1

### Formation of a Collagenous Matrix by Human Neonatal Foreskin Fibroblasts

Human neonatal foreskin fibroblasts (originated at Organogenesis, Inc. Canton, Mass.) were seeded at 5×10⁵ cells/162 cm² tissue culture treated flask (Costar Corp., Cambridge, Mass., cat #3150) and grown in growth medium. The growth medium consisted of: Dulbecco's Modified Eagle's medium (DMEM) (high glucose formulation, without L-glutamine, BioWhittaker, Walkersville, Md.) supplemented with 10% newborn calf serum (NBCS) (HyClone Laboratories, Inc., Logan, Utah) and 4 mM L-glutamine (BioWhittaker, Walkersville, Md.). The cells were maintained in an incubator at 37±1° C. with an atmosphere of 10±1% CO₂. The medium was replaced with freshly prepared medium every two to three days. After 8 days in culture, the cells had grown to confluence, that is, the cells had formed a packed monolayer along the bottom of the tissue culture flask, and the medium was aspirated from the culture flask. To rinse the monolayer, sterile-filtered phosphate buffered saline was added to the bottom of each culture flask and then aspirated from the flasks. Cells were released from the flask by adding 5 mL trypsin-versene glutamine (BioWhittaker, Walkersville, Md.) to each flask and gently rocking to ensure complete coverage of the monolayer. Cultures were returned to the incubator. As soon as the cells were released 5 ml of SBTI (Soybean Trypsin Inhibitor) was added to each flask and mixed with the suspension to stop the action of the trypsin-versene. The cell suspension was removed from the flasks and evenly divided between sterile, conical centrifuge tubes. Cells were collected by centrifugation at approximately 800-1000×g for 5 minutes.

Cells were resuspended using fresh medium to a concentration of 3.0×10⁶ cells/ml, and seeded onto 0.4 micron pore size, 24 mm diameter tissue culture treated inserts (TRANSWELL®, Corning Costar) in a six-well tray at a density of 3.0×10⁶ cells/insert (6.6×10⁵ cells/cm²). The cells were maintained in an incubator at 37±1° C. with an atmosphere of 10±1% CO₂ and fed fresh production medium every 2 to 3 days for 21 days. The production medium comprised: a 3:1 base mixture of DMEM and Hams F-12 medium (Quality Biologics Gaithersburg, Md.), 4 mM GlutaMAX-1™ (Gibco BRL, Grand Island, N.Y.) and additives to a resultant concentration of: 5 ng/ml human recombinant epidermal growth factor (Upstate Biotechnology Lake Placid, N.Y.), 2% newborn calf serum (Hyclone, Logan, Utah), 0.4 μg/ml hydrocortisone (Sigma St. Louis, Mo.), 1×10⁻⁴ M ethanolamine (Fluka, Ronkonkoma, N.Y. ACS grade), 1×10⁻⁴ M o-phosphoryl-ethanolamine (Sigma, St. Louis), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Co., Milwaukee, Wis.), 50 ng/ml L-ascorbic acid (WAKO Chemicals USA, Inc. #013-12061), 0.2 μg/ml L-proline (Sigma, St. Louis, Mo.), 0.1 μg/ml glycine (Sigma, St. Louis, Mo.) and 0.05% poly-ethylene glycol (PEG) 3400-3700 MW (cell culture grade) (Sigma, St. Louis, Mo.).

Samples for histological analysis were taken at days 7, 14 and 21 and fixed in formalin, then embedded in paraffin. The formalin fixed samples were embedded in paraffin and 5 micrometer section were stained with hematoxylin-eosin (H&E) according to procedures known in the art. Using H&E stained slides, thickness measurements were made to ten randomly picked microscopic fields utilizing a 10× eyepiece loaded with a 10 mm/100 micrometer reticle.

Results for two different cell strains of human dermal fibroblasts are summarized in Table 1, which shows the thickness of the cell-matrix construct as it develops.

TABLE 1
Thickness (microns)

| | Day 0 | Day 7 | Day 14 | Day 21 |
|---|---|---|---|---|
| B119 Average | 0 | 30.33 ± 2.61 | 63.33 ± 4.40 | 84.00 ± 4.67 |
| (n = 3) | | | | |
| B156 Average | 0 | 42.00 ± 5.14 | 63.85 ± 4.50 | 76.25 ± 8.84 |
| (n = 4) | | | | |

Samples were also submitted for collagen concentration analysis on days 7, 14, and 21. Collagen content was estimated by employing a calorimetric assay for hydroxyproline content known in the art (Woessner, 1961). At those same timepoints cell number was also determined. Table 2 is a summary of collagen concentration and Table 3 is a summary of the cell data from cell-matrix constructs produced from two different cell strains (B156 and B119) using the procedure described above.

TABLE 2
Collagen (μg/cm²)

| | Day | | | |
|---|---|---|---|---|
| | 0 | Day 7 | Day 14 | Day 21 |
| B119 Average | 0 | 93.69 ± 22.73 | 241.66 ± 21.08 | 396.30 ± 29.38 |
| (n = 3) | | | | |
| B156 Average | 0 | 107.14 ± 17.16 | 301.93 ± 23.91 | 457.51 ± 25.00 |
| (n = 3) | | | | |

TABLE 3
Cells (cells/cm²)

| | Day 0 | Day 7 | Day 14 | Day 21 |
|---|---|---|---|---|
| B119 Average | 6.6 × 10⁵ | 11.8 ± 4.4 × | 11.4 ± 1.7 × | 13.9 ± 1.2 × |
| (n = 3) | | 10⁵ | 10⁵ | 10⁵ |
| B156 Average | 6.6 × 10⁵ | 13.1 ± 0.5 × | 14.0 ± 2.1 × | 17.1 ± 1.7 × |
| (n = 3) | | 10⁵ | 10⁵ | 10⁵ |

Samples of the human cell derived dermal matrix from days 7, 14, and 21 were analyzed by delayed reduction SDS-PAGE to determine collagen composition revealing type I and type III collagen alpha bands in the samples.

Biochemical characteristics of the dermal matrix were determined using immunohistochemical methods. Fibronectin identification was carried out on paraffin fixed sections using the Zymed Histostain strepavidin-biotin system (Zymed Laboratories Inc., South San Francisco, Calif.). Tenascin presence was determined by primary anti-tenascin antibody staining (Dako, Carpintheria, Calif.) followed by anti-mouse horseradish peroxidase labeled antibody (Calbiochem) as a secondary antibody. Samples were visualized by applying diaminobenzyne (Sigma St. Louis, Mo.) and counterstained with Nuclear Fast red.

Glycosaminoglycan (GAG) quantification was performed on 21 day samples using the previously described method (Farndale, 1986). The assay showed the presence of 0.44 grams of GAG per cm² in a sample of human cell derived dermal matrix taken 21 days post seeding.

### Example 2

### In Vitro Formation of a Collagenous Matrix by Human Neonatal Foreskin Fibroblasts in Chemically Defined Medium

Human neonatal foreskin fibroblasts were expanded using the procedure described in Example 1. Cells were then resuspended to a concentration of 3×10⁶ cells/ml, and seeded on to 0.4 micron pore size, 24 mm diameter tissue culture treated membrane inserts in a six-well tray at a density of 3.0×10⁶ cells/TW (6.6×10⁵ cells/cm²). These cells were then maintained as Example 1 with newborn calf serum omitted from the media throughout. More specifically the medium contained: a base 3:1 mixture of DMEM, Hams F-12 medium (Quality Biologics, Gaithersburg, Md.), 4 mM GlutaMAX (Gibco BRL, Grand Island, N.Y.) and additives: 5 ng/ml human recombinant epidermal growth factor (Upstate Biotechnology, Lake Placid, N.Y.), 0.4 μg/ml hydrocortisone (Sigma, St. Louis, Mo.), 1×10⁻⁴ M ethanolamine (Fluka, Ronkonkoma, N.Y. cat. #02400 ACS grade), 1×10⁻⁴ M o-phosphoryl-ethanolamine (Sigma, St. Louis, Mo.), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Company, Milwaukee, Wis.), 50 ng/ml L-ascorbic acid (WAKO Chemicals USA, Inc.), 0.2 μg/ml L-proline (Sigma, St. Louis, Mo.), 0.1 μg/ml glycine (Sigma, St. Louis, Mo.) and 0.05% poly-ethylene glycol (PEG) (Sigma, St. Louis, Mo.). Samples were checked at day 7, 14, and 21 for collagen concentration and cell number using described procedures. Results are summarized in tables 4 (cell number) and 5 (collagen). Samples were also formalin fixed and processed for hematoxylin and eosin staining for light microscope analysis as described in Example 1. Histological evaluation demonstrated that the constructs grown in defined medium was similar to those grown in the presence of 2% newborn calf serum. Samples also stained positively for fibronectin, using procedure described in Example 1.

TABLE 4
Collagen (μg/cm²)

| | Day | | | |
|---|---|---|---|---|
| | 0 | Day 7 | Day 14 | Day 21 |
| Average | 0 | 107.63 ± 21.96 | 329.85 ± 27.63 | 465.83 ± 49.46 |
| amount of | | | | |
| collagen in | | | | |
| each construct | | | | |
| (n = 3) | | | | |

TABLE 5
Cells (cells/cm²)

| | Day 0 | Day 7 | Day 14 | Day 21 |
|---|---|---|---|---|
| Average | 6.6 × 10⁵ | 7.8 ± 2.2 × 10⁵ | 9.6 ± 2.5 × 10⁵ | 1.19 ± 2.1 × |
| number | | | | 10⁵ |
| of cells | | | | |
| in each | | | | |
| construct | | | | |
| (n = 3) | | | | |

Besides endogenously produced fibrillar collagen, decorin and glycosaminoglycan were also present in the cell-matrix construct.

### Example 3

### In Vitro Formation of a Collagenous Matrix by Human Achilles Tendon Fibroblasts

Cell-matrix constructs were formed using the same method described in Example 1 replacing the human neonatal foreskin fibroblasts with human Achilles tendon fibroblasts (HATF.). Following 21 days in production medium, samples were also submitted for H&E staining and thickness determination using the procedure described in Example 1. The resulting construct was visualized as a cell matrix tissue like construct with a thickness of 75.00±27.58 microns (n=2). Endogenously produced fibrillar collagen, decorin and glycosaminoglycan were also present in the construct.

### Example 4

### In Vitro Formation of a Collagenous Matrix by Transfected Human Neonatal Foreskin Fibroblasts

Transfected human dermal fibroblasts were produced using the following procedure. One vial of jCRIP-43 platelet derived growth factor (PDGF) viral producers (Morgan, .J, et al.) was thawed, and the cells were seeded at 2×10⁶ cells/162 cm² flask (Corning Costar, Cambridge, Mass.). These flasks were fed a growth medium, and maintained in an incubator at 37±1° C. with an atmosphere of 10±1% CO₂. The growth medium consisted of: Dulbecco's modified Eagle's medium (DMEM) (high glucose formulation, without L-glutamine, BioWhittaker, Walkersville, Md.) supplemented with 10% newborn calf serum (HyClone Laboratories, Inc., Logan, Utah) and 4 mM L-glutamine (BioWhittaker, Walkersville, Md.). On the same day, 1 vial of human neonatal foreskin fibroblast (HDFB156) was also thawed and plated at 1.5×10⁶ cells/162 cm² flask (Corning Costar, Cambridge, Mass.). After three days the jCRIP PDGF-43 viral producers were fed with fresh growth medium. The HDFB156 were fed with the above growth medium plus 8 μg/ml polybrene (Sigma, St. Louis, Mo.). The next day the HDFB156's cells were infected as follows. The spent medium from the jCRIP PDGF-43 viral producers was collected and filtered through a 0.45 micron filter. 8 μg/ml polybrene was added to this filtered spent medium. The spent medium was then placed on the HDF. On the next two days the HDF were fed fresh growth medium. The day after the HDF were passed from p5 to p6 and seeded at a density of 2.5×10⁶ cells/162 cm² flask (Corning Costar, Cambridge, Mass.). Cells were passed as follows; spent medium was aspirated off. The flasks were then rinsed with a phosphate buffered saline to remove any residual newborn calf serum. Cells were released from the flask by adding 5 mL trypsin-versene to each flask and gently rocking to ensure complete coverage of the monolayer. Cultures were returned to the incubator. As soon as the cells were released, 5 mL of SBTI (Soybean Trypsin Inhibitor) was added to each flask and mixed with the suspension to stop the action of the trypsin-versene. The cell/Trypsin/SBTI suspension was removed from the flasks and evenly divided between sterile, conical centrifuge tubes. Cells were collected by centrifugation at approximately 800-1000×g for 5 minutes). The cells were resuspended in the growth media for seeding at the density listed above. After two days the cells were fed fresh growth medium. The following day the cells were harvested as above, and diluted to a density of 1.5×10⁶ cells/ml in growth medium containing 10% newborn calf serum (NBCS) with 10% dimethyl sulfoxide (DMSO) (Sigma, St. Louis, Mo.). The cells were then frozen 1 ml/cryovial at about −80° C.

Production of the collagenous matrix for this example utilize the same procedure as Examples 1 and 3, replacing the human neonatal foreskin fibroblasts with human neonatal foreskin fibroblasts transformed to produce high levels of platelet derived growth factor (PDGF) as described above. Samples were taken for H&E staining as described above on day 18 post seeding. Samples were also stained using the avidin-biotin methods for the presence of fibronectin listed in Example 10. Samples were taken on day 18 post seeding for H&E staining as described in Example 1, and exhibited a similar cell-matrix gross appearance to that described in Example 1, with a measured thickness of 123.6 microns (N=1). PDGF output of the transfected cells in the cell-matrix construct was measured to be 100 ng/mL by ELISA throughout the duration of the culture (18 days) while control output of PDGF was undetectable.

### Example 5

### In Vitro Formation Of A Matrix By Human Corneal Keratocytes

Human corneal keratocyte cells (originated at Organogenesis, Inc. Canton, Mass.) were used in the production of a stromal construct of cornea. Confluent cultures of human keratocytes were released from their culture substrates using trypsin-versene. When released, soybean trypsin inhibitor was used to neutralize the trypsin-versene, the cell suspension was centrifuged, the supernatant discarded and the cells were then resuspended in base media to a concentration of 3×10⁶ cells/ml. Cells were seeded onto 0.4 micron pore size, 24 mm diameter tissue culture treated transwells in a six-well tray at a density of 3.0×10⁶ cells/TW (6.6×10⁵ cells/cm²). These cultures were maintained overnight in seed medium. The seed medium was composed of: a base 3:1 mixture of Dulbecco's Modified Eagle's Medium (DMEM) and Hams F-12 Medium (Quality Biologics Gaithersburg, Md. cat.), 4 mM GlutaMAX (Gibco BRL, Grand Island, N.Y.) and additives: 5 ng/ml human recombinant epidermal growth factor (EGF) (Upstate Biotechnology Lake Placid, N.Y.), 0.4 μg/ml hydrocortisone (Sigma St. Louis, Mo.), 1×10⁻⁴ M ethanolamine (Fluka, Ronkonkoma, N.Y.), 1×10⁻⁴ M o-phosphoryl-ethanolamine (Sigma, St. Louis, Mo.), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Company, Milwaukee, Wis.). Following this the cultures were fed fresh production medium. The production medium was composed of: a base 3:1 mixture of DMEM, Hams F-12 medium (Quality Biologics Gaithersburg, Md.), 4 mM GlutaMAX (Gibco BRL., Grand Island, N.Y.) and additives: 5 ng/ml Human Recombinant Epidermal growth factor (Upstate Biotechnology Lake Placid, N.Y.), 2% newborn calf serum (Hyclone, Logan, Utah), 0.4 μg/ml hydrocortisone (Sigma, St. Louis, Mo.), 1×10⁻⁴ M ethanolamine (Fluka, Ronkonkoma, N.Y. ACS grade), 1×10⁻⁴ M o-phosphoryl-ethanolamine (Sigma, St. Louis), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Co., Milwaukee, Wis.), 50 ng/ml L-ascorbic acid (WAKO pure chemical company), 0.2 μg/ml L-proline (Sigma, St. Louis, Mo.), 0.1 μg/ml glycine (Sigma, St. Louis, Mo.) and 0.05% poly-ethylene glycol (PEG) (Sigma, St. Louis, Mo., cell culture grade).

The cells were maintained in an incubator at 37±1° C. with an atmosphere of 10%±1% CO₂ and fed fresh production medium every 2-3 days for 20 days (for a total of 21 days in culture. After 21 days in culture, the keratocytes had deposited a matrix layer of about 40 microns in thickness, as measured by the method described in Example 1. Endogenously produced fibrillar collagen, decorin and glycosaminoglycan were also present in the cell-matrix construct.

### Example 6

### In Vitro Formation of a Collagenous Matrix by Human Neonatal Foreskin Fibroblasts Seeded in Production Media

Human neonatal foreskin fibroblasts (originated at Organogenesis, Inc. Canton, Mass.) were seeded at 1×10⁵ cells/0.4 micron pore size, 24 mm diameter tissue culture treated carriers in a six-well tray (TRANSWELL®, Costar Corp. Cambridge, Mass.) and grown in growth medium. The growth medium consisted of: Dulbecco's Modified Eagle's medium (DMEM) (high glucose formulation, without L-glutamine, BioWhittaker, Walkersville, Md.) supplemented with 10% newborn calf serum (HyClone Laboratories, Inc., Logan, Utah) and 4 mM L-Glutamine (BioWhittaker, Walkersville, Md.). The cells were maintained in an incubator at 37±1° C. with an atmosphere of 10±1% CO₂. The medium was replaced every two to three days. After 9 days in culture the medium was aspirated from the culture dish, and replaced with production medium. The cells were maintained in an incubator at 37±1° C. with an atmosphere of 10±1% CO₂ and fed fresh production medium every 2-3 days for 21 days. The production medium was composed of: a base 3:1 mixture of DMEM, Hams F-12 medium (Quality Biologics, Gaithersburg, Md.), 4 mM GlutaMAX (Gibco BRL, Grand Island, N.Y.) and additives: 5 ng/ml human recombinant epidermal growth factor (Upstate Biotechnology, Lake Placid, N.Y.), 2% newborn calf serum (Hyclone, Logan, Utah), 0.4 μg/ml hydrocortisone (Sigma St. Louis, Mo.), 1×10⁻⁴ M ethanolamine (Fluka, Ronkonkoma, N.Y. ACS grade), 1×10⁻⁴ M o-phosphoryl-ethanolamine (Sigma, St. Louis), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Co., Milwaukee, Wis.), 50 ng/ml L-ascorbic acid (WAKO Pure Chemical Company), 0.2 μg/ml L-proline (Sigma, St. Louis, Mo.), 0.1 μg/ml glycine (Sigma, St. Louis, Mo.) and 0.05% poly-ethylene glycol (PEG) (Sigma, St. Louis, Mo., cell culture grade).

Samples were taken at day 21 and fixed in formalin, then embedded in paraffin. The formalin fixed samples were embedded in paraffin and 5 micrometer section were stained with hematoxylin-eosin (H&E) according techniques routinely used in the art. Using H&E stained slides, measurements were made at ten randomly picked microscopic fields utilizing a 10× Eyepiece (Olympus America Inc., Melville, N.Y.) loaded with a 10 mm/100 micrometer reticle (Olympus America Inc., Melville, N.Y.). The constructs created using this method are similar in structure and biochemical composition to those created with Example 1, and have a measured thickness of 82.00±7.64 microns.

### Example 7

### In Vitro Formation of A Collagenous Matrix by Pig Dermal Fibroblasts

Pig Dermal Fibroblasts (originated at Organogenesis, Inc. Canton, Mass.) were seeded at 5×10⁵ cells/162 cm² tissue culture treated flask (Costar Corp., Cambridge, Mass. cat #3150) and grown in growth medium as described below. The growth medium consisted of, Dulbecco's modified Eagle's medium (DMEM) (high glucose formulation, without L-glutamine, BioWhittaker, Walkersville, Md.) supplemented with 10% fetal calf serum (HyClone Laboratories, Inc., Logan, Utah) and 4 mM L-glutamine (BioWhittaker, Walkersville, Md.). The cells were maintained in an incubator at 37±1° C. with an atmosphere of 10%±1% CO₂. The medium was replaced every two to three days. Upon confluence, that is the cells had formed a packed layer at the bottom of the tissue culture flask, the medium was aspirated from the culture dish. To rinse the monolayer, sterile-filtered phosphate buffered saline was added to the monolayer and then aspirated from the dish. Cells were released from the flask by adding 5 ml trypsin-versene glutamine (BioWhittaker, Walkersville, Md.) to each flask and gently rocking to ensure complete coverage of the monolayer. Cultures were returned to the incubator. As soon as the cells were released 5 ml of SBTI (Soybean Trypsin Inhibitor) was added to each flask and mixed with the cell suspension to stop the action of the trypsin-versene. The suspension was removed from the flasks and evenly divided between sterile, conical centrifuge tubes. Cells were collected by centrifugation at approximately 800-1000×g for 5 minutes. Cells were resuspended and diluted to a concentration of 3×10⁶ cells/ml, and seeded onto 0.4 micron pore size, 24 mm diameter tissue culture treated transwells in a six-well tray at a density of 3.0×10⁶ cells/TW (6.6×10⁵ cells/cm²). Cells were maintained overnight in a seed medium. The seed medium consisted of, a base 3:1 mixture of DMEM, Hams F-12 medium (Quality Biologics, Gaithersburg, Md.), 4 mM GlutaMAX (Gibco BRL, Grand Island, N.Y.) and additives: 5 ng/ml human recombinant epidermal growth factor (Upstate Biotechnology Lake Placid, N.Y.), 0.4 μg/ml hydrocortisone (Sigma St. Louis, Mo.), 1×10⁻⁴ M ethanolamine (Fluka, Ronkonkoma, N.Y. ACS grade), 1×10⁻⁴ M o-phosphoryl-ethanolamine (Sigma, St. Louis), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Co., Milwaukee, Wis.), 50 ng/ml L-ascorbic acid (WAKO Pure Chemical Company), 0.2 μg/ml L-proline (Sigma, St. Louis, Mo.), and 0.1 μg/ml glycine (Sigma, St. Louis, Mo.). The cells were maintained in an incubator at 37±1° C. with an atmosphere of 10±1% CO₂ and fed fresh production medium every 2-3 days for 7 days. The production medium was composed of: a base 3:1 mixture of DMEM, Hams F-12 medium (Quality Biologics, Gaithersburg, Md.), 4 mM GlutaMAX (Gibco BRL, Grand Island, N.Y.) and additives: 5 ng/ml human recombinant epidermal growth factor (Upstate Biotechnology, Lake Placid, N.Y.), 2% newborn calf serum (Hyclone, Logan, Utah), 0.4 μg/ml hydrocortisone (Sigma St. Louis, Mo.), 1×10⁻⁴ M ethanolamine (Fluka, Ronkonkoma, N.Y. ACS grade), 1×10⁻⁴ M o-phosphoryl-ethanolamine (Sigma, St. Louis), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Co., Milwaukee, Wis.), 50 ng/ml L-ascorbic acid (WAKO Pure Chemical Company), 0.2 μg/ml L-proline (Sigma, St. Louis, Mo.), 0.1 μg/ml glycine (Sigma, St. Louis, Mo.) and 0.05% poly-ethylene glycol (PEG) (Sigma, St. Louis, Mo.) cell culture grade. After 7 days the media was replaced with production medium without newborn calf serum. This media was fed fresh to the cells every 2-3 days for 20 more days, for a total of 28 days in culture.

Samples were taken at day 21 and fixed in formalin, then embedded in paraffin. The formalin fixed samples were embedded in paraffin and 5 micrometer section were stained with hematoxylin-eosin (H&E) according to techniques customarily used in the art. Using H&E stained slides, measurements were made at ten randomly picked microscopic fields utilizing a 10× Eyepiece (Olympus America Inc., Melville, N.Y.) loaded with a 10 mm/100 micrometer reticle (Olympus America Inc., Melville, N.Y.). The sample exhibited a structure composed of cells and matrix with a measured thickness of 71.20±9.57 microns. Besides endogenously produced fibrillar collagen, decorin and glycosaminoglycan were also present in the cell-matrix construct.

### Example 8

### In Vitro Formation of a Collagenous Matrix by Human Neonatal Foreskin Fibroblasts in Chemically Defined Medium

Human neonatal foreskin fibroblasts were expanded using the procedure described in Example 1. Cells were then resuspended to a concentration of 3×10⁶ cells/ml, and seeded on to 0.4 micron pore size, 24 mm diameter tissue culture treated membrane inserts in a six-well tray at a density of 3.0×10⁶ cells/TW (6.6×10⁵ cells/cm²). Cells in this example were cultured in chemically defined medium throughout.

The medium contained: a base 3:1 mixture of DMEM, Hams F-12 medium (Quality Biologics, Gaithersburg, Md.), 4 mM GlutaMAX (Gibco BRL, Grand Island, N.Y.) and additives: 5 ng/ml human recombinant epidermal growth factor (Upstate Biotechnology, Lake Placid, N.Y.), 1×10⁻⁴ M ethanolamine (Fluka, Ronkonkoma, N.Y. cat. #02400 ACS grade), 1×10⁻⁴ M o-phosphoryl-ethanolamine (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Company, Milwaukee, Wis.), 50 ng/ml L-ascorbic acid (WAKO Chemicals USA, Inc.), 0.2 μg/ml L-proline (Sigma, St. Louis, Mo.), 0.1 μg/ml glycine (Sigma, St. Louis, Mo.).

To the basic medium above, other components were added in these separate Conditions:
1. 5 μg/ml insulin (Sigma, St. Louis, Mo.), 0.4 μg/ml hydrocortisone (Sigma, St. Louis, Mo.), 0.05% poly-ethylene glycol (PEG) (Sigma, St. Louis, Mo.).
2. 5 μg/ml insulin (Sigma, St. Louis, Mo.), 0.4 μg/ml hydrocortisone (Sigma, St. Louis, Mo.).
3. 375 μg/ml insulin (Sigma, St. Louis, Mo.), 6 μg/ml hydrocortisone (Sigma, St. Louis, Mo.).

Samples were formalin fixed and processed for hematoxylin and eosin staining for light microscope analysis. Visual histological evaluation demonstrated that the Condition 2 lacking PEG demonstrated a comparably similar matrix as Condition 1 containing PEG. Biochemical analysis measuring the collagen content of the construct showed nearly the same amount of collagen in both: 168.7±7.98 μg/cm² for Condition 1 with PEG as compared to 170.88±9.07 μg/cm² for Condition 2 without PEG. Condition 3 containing high levels of insulin and hydrocortisone showed a higher expression of matrix, including collagen, at a timepoint earlier than the other two conditions. Besides endogenously produced fibrillar collagen, decorin and glycosaminoglycan were also present in the cell-matrix constructs in all Conditions. The cultured dermal construct formed by the method of Condition 2 of this Example is shown in FIG. 2. Shown in FIG. 2 is a photomicrograph of a fixed, paraffin embedded, hematoxylin and eosin stained section of a cell-matrix construct formed from cultured human dermal fibroblasts in chemically defined medium at 21 days. The porous membrane appears as a thin translucent band below the construct and it can be seen that the cells grow on the surface of the membrane and do not envelope in integrate the membrane with matrix.

FIG. 3 shows transmission electron microscope (TEM) images of two magnifications of cultured dermal construct formed by the method of Condition 2 of this Example at 21 days. FIG. 3A is a 7600× magnification showing alignment of endogenous collagen fibers between the fibroblasts. FIG. 3B is a 19000× magnification of fully formed endogenous collagen fibers demonstrating fibril arrangement and packing.

In all Conditions of this Example, the cultured dermal constructs formed comprise dermal fibroblasts and endogenously produced matrix. All have fully formed collagen fibrils in packed organization arranged between the cells. Their fibrous qualities, thickness, and cohesive integrity give the construct considerable strength to allow it to be peelably removed from the culture membrane and handled as it is transferred to a subject to be treated with the construct, as in a graft or implant.

### Example 9

### Formation of a Collagenous Matrix by Human Buccal Fibroblasts

The purpose of this experiment is to produce a cell-matrix construct from buccal fibroblasts isolated from human cheek tissue. Buccal were cultured in T-150 flasks in DMEM containing 10% NBCS medium. After 7 days, to expand the number of cells further, buccal cells were harvested and passaged into nine T-150 flasks at 4.0×10⁶ cells in DMEM containing 10% NBCS medium and cultured until confluence at which time the cells were harvested.

To harvest the cells, the medium was aspirated from the culture flask. To rinse the monolayer, sterile-filtered phosphate buffered saline was added to the bottom of each culture flask and then aspirated from the flasks. Cells were released from the flask by adding 5 mL trypsin-versene glutamine (BioWhittaker, Walkersville, Md.) to each flask and gently rocking to ensure complete coverage of the monolayer. Cultures were returned to the incubator. As soon as the cells were released 5 ml of SBTI (Soybean Trypsin Inhibitor) was added to each flask and mixed with the suspension to stop the action of the trypsin-versene. The cell suspension was removed from the flasks and evenly divided between sterile, conical centrifuge tubes. Cells were collected by centrifugation at approximately 800-1000×g for 5 minutes.

Cells were resuspended using fresh medium to a concentration of 3.0×10⁶ cells/ml, and seeded onto 0.4 micron pore size, 24 mm diameter tissue culture treated inserts (TRANSWELL®, Corning Costar) in a six-well tray at a density of 3.0×10⁶ cells/insert (6.6×10⁵ cells/cm²). The cells were maintained in an incubator at 37±1° C. with an atmosphere of 10±1% CO₂ and fed medium containing: a base 3:1 mixture of DMEM, Hams F-12 medium (Quality Biologics, Gaithersburg, Md.), 4 mM GlutaMAX (Gibco BRL, Grand Island, N.Y.) and additives: 5 ng/ml human recombinant epidermal growth factor (Upstate Biotechnology, Lake Placid, N.Y.), 0.4 μg/ml hydrocortisone (Sigma, St. Louis, Mo.), 1×10⁻⁴ M ethanolamine (Fluka, Ronkonkoma, N.Y. cat. #02400 ACS grade), 1×10⁻⁴ M o-phosphoryl-ethanolamine (Sigma, St. Louis, Mo.), 5 μg/ml insulin (Sigma, St. Louis, Mo.), 5 μg/ml transferrin (Sigma, St. Louis, Mo.), 20 ρM triiodothyronine (Sigma, St. Louis, Mo.), and 6.78 ng/ml selenium (Sigma Aldrich Fine Chemicals Company, Milwaukee, Wis.), 50 ng/ml L-ascorbic acid (WAKO Chemicals USA, Inc.), 0.2 μg/ml L-proline (Sigma, St. Louis, Mo.), 0.1 μg/ml glycine (Sigma, St. Louis, Mo.) and 0.05% poly-ethylene glycol (PEG) (Sigma, St. Louis, Mo.).

At day 1 post seeding, medium was replaced with Serum Free Production Media, exchanged every 2-3 days for 21 days. At day 21, samples were fixed in formalin for histology. Three samples were used for protein and collagen production analysis.

Collagen production for 24 mm diameter constructs averaged 519 μg per construct after 21 days in culture. Total protein production for 24 mm diameter constructs averaged 210 μg per construct after 21 days in culture. Morphologically, the buccal fibroblast cell-matrix construct, a cultured tissue construct of oral connective tissue, showed buccal fibroblasts surrounded by matrix while physically, the construct had physical bulk and integrity.

### Example 10

### Methods for Termination of Fibroblasts in Endogenously Produced Matrix Constructs to Form Devitalized Cell-Matrix Constructs

Termination of fibroblasts in endogenously produced cell-matrix constructs were assayed using the alamarBlue™ assay. The alamarBlue™ assay incorporates an oxidation-reduction indicator that changes in color in response to chemical reduction of growth medium as a direct result from cell metabolism. Metabolic activity from the cells will result in the reduction of alamarBlue™ to a reddish or pink color as exposure time is increased. Lack of viable cells should result in little or no color change.

Twelve 24 mm diameter matrix constructs made according to the method of Example 1 (cell-matrix construct) were grown on 24 mm culture inserts having 0.4 micron pores with each culture insert residing in a deep-well tray for 25 days. Four methods of fibroblast termination (n=3 for each condition) were carried out overnight: (1) air-drying at ambient room temperature and humidty; (2) rinsing in 100% ethanol; (3) snap freezing followed by air drying; and, (4) lyophilization. The 24 mm membrane culture inserts were transferred back to the original 6-well plates. Each well contained 11.0 mL of Dulbecco's modified Eagle's medium (DMEM) (high glucose formulation, without L-glutamine, BioWhittaker, Walkersville, Md.) with 10% AlamarBlue™. Media was sampled from each well at 8 hours and 24 hours in 200 μL samples, which were taken and aliquotted into a 96-well plate and stored at 2-8° C. After the second time point sample was taken, the samples in the 96-well plate were read on a plate reader at two wavelengths. The percent of AlamarBlue™ reduction was calculated from the absorbance values given by the plate reader.

After 24 hours of incubation, all conditions tested did not show significant metabolic activity suggesting that the cells in each cell-matrix construct had successfully been terminated using these termination methods. The results of this example were devitalized single-layer matrix constructs that may be used in the fabrication of multilayer, or tubular, or complex bioengineered constructs of the invention. The devitalization methods of this example may be applied to any of the living matrix constructs of the previous examples to arrive at a devitalized matrix construct.

### Example 11

### Crosslinking of Human Cell Derived Dermal Matrix

Two 75 mm diameter cell-matrix constructs made according to the method of Example 1 by culturing them on 75 mm diameter, 0.4 micron porous polycarbonate membranes. One cell-matrix construct was devitalized by air drying at room temperature and humidity and the other was devitalized by immersing it overnight in 100% ethanol and then air-drying to allow the ethanol to evaporate from the cell-matrix construct. Both cell-matrix constructs were then rehydrated using a volume of sterile water for injection. To form a 2-layer construct, the cell-matrix constructs were peeled from their respective membranes using curved tweezers and superimposed by layering onto each other using on a 100 mm culture dish lid. Approximately 15 mL of 1.0 mM EDC solution was added to the layered cell-matrix construct and allowed to cross-link overnight, or approximately 15-18 hours. The 2-layer construct was removed from the EDC solution, rinsed in sterile water and air-dried to result in a 2-layer, crosslinked, devitalized cell-matrix construct, endogenously produced by human fibroblast cells.

The construct was then re-hydrated. When rehydrated, the 2-layer construct quickly gained water mass and was smooth in texture. The rehydration process was nearly instantaneous, taking under a minute. The 2-layer construct looked and handled as a single piece of tissue. The construct was cut into four strips roughly 1.25×5 cm then tested on an Instron mechanical testing machine for strength. The average strength for each strip recorded by the Instron machine was 4.9 N.

### Example 12

### Layering and Cross-Linking Devitalized Tissue Constructs to Form Multilayered Constructs

Sixteen matrix constructs of were grown on 75 mm diameter culture insert membranes having pores of about 0.4 μm using methods and materials substantially similar to Example 1. To devitalize them, the tissue constructs were air-dried overnight in ambient room temperature and humidity to ensure fibroblast termination. The devitalized tissue constructs were then hydrated with water for injection (WFI) and the tissue constructs were peeled from each membrane using curved tweezers or blunt forceps. A devitalized tissue construct was fully spread out onto 100 mm culture dish lids (as a convenient working surface) and subsequent layers were added by superimposing the devitalized tissue constructs on top of one another to achieve 2 or 3 layers. The layers were allowed to air-dry in ambient room temperature and humidity overnight, or between about 15-18 hours. After drying, the superimposed layers adhered to each other. The adhered layers were hydrated and then superimposed on other adhered layers and then again allowed to dry overnight, or between about 15-18 hours. From the sixteen single layer devitalized tissue constructs, a 10-layer, a 5-layer and a single layer construct were formed with the layers of the 10-layer and 5-layer constructs adhered to each other by the drying and rehydration process. The constructs were then crosslinked with a crosslinking agent. The constructs were in vessels and to each vessel was added approximately 10 mL of 1.0 mM EDC in WFI solution. The layered devitalized ECM tissue constructs were allowed to cross-link overnight (approximately between 15-18 hours). The tissue constructs were removed from the EDC solution, rinsed with WFI and air-dried in ambient room temperature and humidity one final time resulting in a crosslinked, multilayer, devitalized human cell-derived tissue construct.

The two multi-layered constructs were cut into three strips roughly 1.25×5 cm then tested on an Instron mechanical testing machine to measure strength. The normalized tensile strength per layer of the 5 layer tissue construct was determined to be 1.425 N/layer; for the 10 layer tissue construct the tensile strength was 1.706 N/layer.

The combination of air-drying, layering, and EDC cross-linking the non-cellular ECM tissue constructs was successful at giving a strength and uniformity to the non-cellular ECM. Non-cellular ECM tissue constructs can successfully be layered, dried and rehydrated. The handling and shape of the non-cellular ECM tissue constructs did not diminish from multiple drying and rehydration steps. Increasing the number of non-cellular ECM tissue construct layers to five and as many as ten increased the overall strength of the construct as well as maintained the shape of the tissue after being cut to size.

### Example 13

### Fabrication of Multi-Layer Cell-Matrix Constructs Crosslinked Using Transglutaminase or Transglutaminase/EDC

Devitalized cell-matrix constructs were layered and crosslinked using transglutaminase or transglutaminase followed with EDC in MES buffer to bond the layers together. Transglutaminase (“TGM”) is a collective term for a family of naturally occurring enzymes that act to link proteins. TGM catalyzes covalent bond formation between free amine groups and γ-carboxamide group specifically acting on lysine and glutamine. There are several forms of TGM, but the type focused on in this experiment will be Activa™ food-grade TGM, which is made through a fermentation process and is used as a binding agent. It comes ready to use a dry powder but can also be mixed as a slurry or solution.

Twelve cell-matrix constructs were grown in 75 mm diameter culture inserts having 0.4 μm porous membranes and cultured for 25 days according methods and using materials substantially similar to those presented in Example 1. The cell-matrix constructs were devitalized by air-drying them overnight, approximately 15-18 hours, at ambient room temperature and humidity. The dried, devitalized cell-matrix constructs were hydrated with sterile water for injection (WFI) and the constructs were peeled from their respective membrane using blunt forceps. Cell-matrix constructs were fully spread out onto a 100 mm culture dish with superimposed cell-matrix constructs layered over them to form three 4-layer constructs. When layering, each cell-matrix layer was contacted with 5.0 mL TGM solution of varying concentration (the concentrations for each 4-layer construct were 28U, 280U, and 700U) added in between individual layers to ensure that the entire cell-matrix layer was treated with crosslinking agent. The cell-matrix constructs were layered loosely in cross-linking solution to evaluate fusing of layers without application of pressure or a drying step. The three 4-layer cell-matrix constructs were placed into a 40° C. incubator overnight, or approximately 15-18 hours. The three 4-layer cell-matrix constructs were removed from the 40° C. incubator and allowed to air-dry at ambient room temperature and humidity. After fully air-drying, the cell-matrix constructs were hydrated with sterile WFI. The cell-matrix constructs were cut in half with half of each construct then treated with 1.0 mM EDC in MES buffer and allowed to cross-link overnight at 4° C. The other halves of each construct that were crosslinked with TGA alone were also stored overnight at 4° C. in WFI. The following day all 6 pieces were tested for tensile strength and suture retention on the Instron machine.

Although results varied from sample to sample, generally, all conditions yielded 4-layer, bonded constructs of devitalized endogenously produced cell-matrix layers having comparable strength per layer measures when comparing TGA treatment alone against TGA/EDC crosslinked and bonded constructs.

### Example 14

### Fabrication of Multi-Layer Cell-Matrix Constructs Crosslinked Using Food-Grade Transglutaminase or Recombinant Transglutaminase

Two forms of transglutaminase (TGM) on multilayered, devitalized cell-matrix constructs were compared. The forms of the enzyme compared were Activa™ food-grade and recombinant human transglutaminase.

Thirty-two cell-matrix constructs were grown in 75 mm diameter culture inserts having 0.4 μm porous membranes and cultured for 25 days according to methods and using materials substantially similar to those presented in Example 1. All cell-matrix constructs were devitalized by air drying in ambient room temperature and humidity. The dried, devitalized single-layer cell-matrix constructs were hydrated 24 hours later with sterile water for injection (WFI) and the constructs were then peeled from their respective membranes using blunt forceps. Cell-matrix constructs having eight layers were then fabricated.

The cell-matrix sheets were layered in pairs by superimposing them to yield sixteen 2-layer cell-matrix constructs. These were again allowed to air-dry in ambient room temperature and humidity in aseptic conditions. Four sets of 2-layered constructs were spread out onto a 100 mm plate and each had 20 mL of a specific type and activity of TGM or rhTGM solutions was added: (1) 8-layer food-grade TGM 5U; (2) 8-layer food-grade TGM 10U; (3) 8-layer rhTGM 15U; and, (4) 8-layer rhTGM 30U. The constructs were placed into a 40° C. incubator and allowed to crosslink overnight. The following day, each condition of 2-layered constructs was layered into 4-layer constructs. This process was repeated again but without TGM to achieve one 8-layer construct for each crosslinking condition. These were air-dried in ambient room temperature and humidity and treated with the same TGM crosslinking agent for a second and final time. The constructs were placed into a 40° C. incubator and allowed to crosslink overnight, approximately 18-24 hours. After a final air-drying, the constructs were hydrated with sterile WFI. The thickness of each construct was measured using a laser. Three pieces from each 8-layered unit were tested for tensile strength and suture retention on the Instron machine.

Cross-linking with rhTGM was more successful than food-grade TGM for 8-layered constructs in this experiment. The lower concentration of rhTGM resulted in better tensile strength and suture retention than other conditions.

### Example 15

### Connective Tissue Construct

Fibroblasts were seeded retrieved from scale-up cultures where fibroblasts were cultured on microcarriers in bioreactors. Fibroblasts were strained using a stainless steel sieve set-up to separate the fibroblasts from the microcarriers. This removed all microcarriers and cell clumps from the cell suspension. Approximately 3.0×10⁷ cells were seeded to a 0.4 micron porous membrane of approximately 44 cm² in surface area bathed in about 140 mL chemically defined matrix production medium. This seeding density was at super-confluence.

The chemically defined matrix production medium contained a base of DMEM (high glucose, without L-glutamine) supplemented with approximate amounts of the following: 4 mM L-glutamine; 10 ng/ml human recombinant epidermal growth factor; 1×10⁻⁴ M ethanolamine; 1×10⁻⁴ M o-phosphoryl-ethanolamine; 5 μg/ml transferrin, 20 ρM triiodothyronine, 5 μg/ml insulin; 6.78 ng/ml selenious acid; 50 ng/ml magnesium ascorbate; 0.2 μg/ml L-proline; 0.1 μg/ml glycine; 0.02 μg/ml human recombinant long chain TGF-alpha; 0.0038 μg/ml prostaglandin E₂ (PGE2); 0.4 μg/ml hydrocortisone. Matrix production medium was exchanged with fresh matrix production medium every 3-4 days for 18 days. During this time, an endogenous cell-matrix construct had formed by the cells.

### Example 16

### Bilayer Skin Construct

A skin construct having a fibroblast layer and a keratinocyte layer was formed in a fully chemically defined culture media system. Fibroblasts were seeded retrieved from scale-up cultures where fibroblasts were cultured on microcarriers in bioreactors. Fibroblasts were strained using a stainless steel sieve set-up to separate the fibroblasts from the microcarriers. This removed all microcarriers and cell clumps from the cell suspension. Approximately 1.0×10⁷ cells were seeded to a 0.4 micron porous membrane of approximately 44 cm² in surface area bathed in about 130 mL chemically defined matrix production medium. This seeding density was at super-confluence.

The chemically defined matrix production medium contained:

| Component | Concentration | |
|---|---|---|
| DMEM | 96.0% | |
| L-Glutamine | 1060 | mg/L |
| Hydrocortisone | 0.4 | mg/L |
| Selenious acid | 6.78 | μg/L |
| Ethanolamine | 0.1 | mM |
| o-Phosphorylethanolamine | 14.0 | Mg/L |
| EGF | 10.0 | μg/L |
| Mg Ascorbate | 50 | mg/L |
| L-Proline | 213.6 | mg/L |
| Glycine | 101.4 | mg/L |
| Long TGFα | 10.0 | μg/L |

Fibroblasts were cultured in the matrix production medium for 11 days with media changes made periodically, every 3-4 days.

At day 11, a suspension of keratinocytes was seeded onto the surface of the cell-matrix construct at an approximate density of 3.3×10⁶ cells in a medium containing approximately:

| Component | Concentration | |
|---|---|---|
| DMEM:HAM's F-12 3:1 | 96.10% | |
| L-Glutamine | 1060 | mg/L |
| Hydrocortisone | 0.4 | mg/L |
| Insulin | 5.0 | mg/L |
| Transferrin | 5.0 | mg/L |
| Triiodothyronine | 13.5 | ng/L |
| Ethanolamine | 0.1 | mM |
| o-Phosphorylethanolamine | 14.0 | Mg/L |
| Selenious acid | 6.78 | μg/L |
| Adenine | 24.4 | mg/L |
| Mg Ascorbate | 50.0 | mg/L |
| Progesterone | 0.63 | μg/L |
| EGF | 10.0 | μg/L |
| Long TGFα | 10.0 | μg/L |
| Lipid Concentrate | | |
| Arachidonic Acid | 0.004 | mg/L |
| Cholesterol | 0.220 | mg/L |
| DL-α-Tocopherol- | 0.140 | mg/L |
| Acetate | | |
| Linoleic Acid | 0.020 | mg/L |
| Linolenic Acid | 0.020 | mg/L |
| Myristic Acid | 0.020 | mg/L |
| Oleic Acid | 0.020 | mg/L |
| Palmitoleic Acid | 0.020 | mg/L |
| Palmitic Acid | 0.020 | mg/L |
| Pluronic ® F-68 | 200.0 | mg/L |
| Stearic Acid | 0.020 | mg/L |
| Tween ® 80 | 4.4 | mg/L |

At day 13, differentiation was induced by adding use of a differentiation medium containing the following:

| Component | Concentration | |
|---|---|---|
| DMEM:HAM's F-12 3:1 | 96.3% | |
| L-Glutamine | 1060 | mg/L |
| Hydrocortisone | 0.40 | mg/L |
| Insulin | 5.0 | mg/L |
| Transferrin | 5.0 | mg/L |
| Triiodothyronine | 13.5 | ng/L |
| Selenious acid | 0.00678 | mg/L |
| Ethanolamine | 0.1 | mM |
| o-Phosphorylethanolamine | 14.0 | Mg/L |
| Adenine | 24.4 | mg/L |
| Mg Ascorbate | 50.0 | mg/L |
| Progesterone | 0.63 | μg/L |
| CaCl2 | 265 | mg/L |
| Lipid Concentrate | | |
| Arachidonic Acid | 0.004 | mg/L |
| Cholesterol | 0.220 | mg/L |
| DL-α-Tocopherol- | 0.140 | mg/L |
| Acetate | | |
| Linoleic Acid | 0.020 | mg/L |
| Linolenic Acid | 0.020 | mg/L |
| Myristic Acid | 0.020 | mg/L |
| Oleic Acid | 0.020 | mg/L |
| Palmitoleic Acid | 0.020 | mg/L |
| Palmitic Acid | 0.020 | mg/L |
| Pluronic ® F-68 | 200.0 | mg/L |
| Stearic Acid | 0.020 | mg/L |
| Tween ® 80 | 4.4 | mg/L |

At day 15, the medium formulation was changed to induce cornification of the developing keratinocyte layer in a medium containing approximately:

| Component | Concentration | |
|---|---|---|
| DMEM | 48.0% | |
| HAM's F-12 | 48.0% | |
| L-Glutamine | 658 | mg/L |
| Hydrocortisone | 0.4 | mg/L |
| Insulin | 5.0 | mg/L |
| Transferrin | 5.0 | mg/L |
| Triiodothyronine | 13.5 | ng/L |
| Ethanolamine | 0.1 | mM |
| o-Phosphorylethanolamine | 14.0 | Mg/L |
| Selenius acid | 6.78 | μg/L |
| Adenine | 24.4 | mg/L |
| Mg Ascorbate | 50.0 | mg/L |
| Long TGFα | 10.0 | μg/L |
| MEM Non-Essential Amino Acid Solution | | |
| L-Alanine | 1.78 | mg/L |
| L-Asparagine | 2.64 | mg/L |
| L-Aspartic Acid | 2.66 | mg/L |
| L-Glutamic Acid | 2.94 | mg/L |
| Glycine | 1.5 | mg/L |
| L-Proline | 2.3 | mg/L |
| L-Serine | 2.1 | mg/L |
| MEM Vitamin Solution | | |
| NaCl | 17 | mg/L |
| D-Ca | 0.2 | mg/L |
| Pantothenate | | |
| Choline | 0.2 | mg/L |
| Chloride | | |
| Folic Acid | 0.2 | mg/L |
| i-Inositol | 0.4 | mg/L |
| Nicotinamide | 0.2 | mg/L |
| Pyridoxal HCl | 0.2 | mg/L |
| Riboflavin | 0.020 | mg/L |
| Thiamine HCl | 0.2 | mg/L |
| Lipid Concentrate | | |
| Arachidonic | 0.004 | mg/L |
| Acid | | |
| Cholesterol | 0.220 | mg/L |
| DL-α- | 0.140 | mg/L |
| Tocopherol- | | |
| Acetate | | |
| Linoleic Acid | 0.020 | mg/L |
| Linolenic Acid | 0.020 | mg/L |
| Myristic Acid | 0.020 | mg/L |
| Oleic Acid | 0.020 | mg/L |
| Palmitoleic Acid | 0.020 | mg/L |
| Palmitic Acid | 0.020 | mg/L |
| Pluronic ® F-68 | 200.0 | mg/L |
| Stearic Acid | 0.020 | mg/L |
| Tween ® 80 | 4.4 | mg/L |

Cornification medium was changed every 2-3 days.

Skin constructs matured and maintained during days 22 through 35 and were fed a maintenance medium with changes every 2-3 days with fresh maintenance medium containing:

| Component | Concentration | |
|---|---|---|
| DMEM | 48.0% | |
| HAM's F-12 | 48.0% | |
| L-Glutamine | 658 | mg/L |
| Hydrocortisone | 0.4 | mg/L |
| Insulin | 5.0 | mg/L |
| Transferrin | 5.0 | mg/L |
| Triiodothyronine | 13.5 | ng/L |
| Ethanolamine | 0.1 | mM |
| O-phosphorylethanolamine | 14.0 | mg/L |
| Selenius acid | 6.78 | μg/L |
| Adenine | 24.4 | mg/L |
| Long TGFα | 10.0 | μg/L |
| MEM Non-Essential Amino Acid Solution | | |
| L-Alanine | 1.78 | mg/L |
| L-Asparagine | 2.64 | mg/L |
| L-Aspartic Acid | 2.66 | mg/L |
| L-Glutamic Acid | 2.94 | mg/L |
| Glycine | 1.5 | mg/L |
| L-Proline | 2.3 | mg/L |
| L-Serine | 2.1 | mg/L |
| MEM Vitamin Solution | | |
| NaCl | 17 | mg/L |
| D-Ca | 0.2 | mg/L |
| Pantothenate | | |
| Choline | 0.2 | mg/L |
| Chloride | | |
| Folic Acid | 0.2 | mg/L |
| i-Inositol | 0.4 | mg/L |
| Nicotinamide | 0.2 | mg/L |
| Pyridoxal HCl | 0.2 | mg/L |
| Riboflavin | 0.020 | mg/L |
| Thiamine HCl | 0.2 | mg/L |
| Lipid Concentrate | | |
| Arachidonic | 0.004 | mg/L |
| Acid | | |
| Cholesterol | 0.220 | mg/L |
| DL-α- | 0.140 | mg/L |
| Tocopherol- | | |
| Acetate | | |
| Linoleic Acid | 0.020 | mg/L |
| Linolenic Acid | 0.020 | mg/L |
| Myristic Acid | 0.020 | mg/L |
| Oleic Acid | 0.020 | mg/L |
| Palmitoleic Acid | 0.020 | mg/L |
| Palmitic Acid | 0.020 | mg/L |
| Pluronic ® F-68 | 200.0 | mg/L |
| Stearic Acid | 0.020 | mg/L |
| Tween ® 80 | 4.4 | mg/L |

When fully formed the cultured skin constructs exhibited a cell-matrix layer of endogenously produced extracellular matrix and its fibroblasts with a differentiated keratinocyte layer disposed atop the cell-matrix layer.

### Example 17

### Fabrication of Collagenous Material-Synthetic Polymer Constructs Using Silk Fibroin Adhesive Solution

Adhesion properties of a purified xenogeneic collagen layer (e.g. “ICL”) with various objects were tested. More particularly, without having the intention of being limited to the following embodiments, adhesion properties between (a) two ICL layers; (b) at least one ICL layer and at least one polyhydroxyalkanoates- (PHA)-based polymeric frame; and (c) at least one ICL layer to at least one PHA-based polymeric rod were analyzed. Preparation of the samples are described in the following examples:
(a) A dried single layer ICL was cut into pieces of 1×2.5 cm in size. Two pieces of ICL was attached with an overlapping size of 0.5 cm and a drop of silk solution was applied between the layers. It is to be appreciated that the dimensions of the cut ICL pieces, and the overlapping size, are not limited to the above examples.
(b) A PHA polymeric-based frame was dipped in silk fibroin solution for at least one minute to coat the frame surface struts with a thin layer of the silk fibroin solution. A piece of ICL (about 1.5×1.5 cm) was then attached to the leading end of the frame.
(c) A PHA polymeric-based rod was dipped in silk fibroin solution for at least one minute in an amount sufficient to coat the surface of the rod with a thin layer of silk fibroin solution. A piece of ICL (about 1×1.5 cm) was then attached to the rod.

Subsequently, all the samples were dried at room temperature for about 1.5 hours. The samples were then immersed in a methanol-based solvent for 5 to 10 minutes, and subsequently dried for 10 minutes. The above samples were soaked in deionized water for at least 24 hours. Adhesion properties achieved by the silk fibroin solution are then evaluated by introducing mechanical strain to the samples using procedures known to one of ordinary skill in the art.

Without having the intention of being limited, it is to be appreciated that employing the silk fibroin aqueous solution as an adhesive can be implemented, for example, between ICL and electrospun collagen, electrospun fibrin, metallic-based materials, ceramic-based materials, tissue-engineered constructs, synthetic polymer materials, natural materials.

### Example 18

### Fabrication of Multi-Layer Cell-Matrix Constructs Using Silk Fibroin Adhesive Solution

In order to make multi-layer cell-tissue constructs, units were first removed from culture, growth and/or matrix production media were aspirated, and the units were allowed to air dry at least until the cells were devitalized. The units were then rehydrated in 10 ml of WFI for about 10 minutes. Additionally and/or alternatively, the units were rehydrated in 5 mL or exposed to about 0.5 mL of about an 8% silk solution, and the units were layered together. The units are then dried overnight, and then subsequently dipped in 3 mL of methanol for at least 10 minutes. The above steps can be repeated until 5 or more layer cell-tissue constructs are formed. As an alternative, it is to be appreciated that transglutaminase can be applied between the layers of the multi-layer cell tissue-constructs. Additionally, after the step of treating the layers with the 8% silk solution, the units can be rolled flat using a roller.

The 5 or more layer cell-tissue constructs can subsequently be lyophilized by placing the constructs in a lyophilizer for at least about 17 hours. It is to be appreciated that the silk solution serves as an adhesive to prevent the layered construct from delamination.

Although the foregoing invention has been described in some detail by way of illustration and Examples for purposes of clarity and understanding, it will be obvious to one of skill in the art that certain changes and modifications may be practiced within the scope of the appended claims.

We claim:

1. A bioengineered construct comprising a devitalized layer of extracellular matrix produced and assembled by cultured extracellular matrix producing cells.

2. The bioengineered construct of claim 1, further comprising one or more additional devitalized layers of extracellular matrix produced and assembled by cultured extracellular matrix producing cells.

3. The bioengineered construct of claim 2, wherein the layers of extracellular matrix have been decellularized of the cultured extracellular matrix producing cells.

4. The bioengineered construct of claim 3, wherein the layers of extracellular matrix adjacently contact each other at a bonding region.

5. The bioengineered construct of claim 4, wherein the layers of extracellular matrix in adjacent contact at the bonding region are bonded together by crosslinks.

6. The bioengineered construct of claim 4, wherein the layers of extracellular matrix in adjacent contact at the bonding region are bonded together by a bioremodelable or bioresorbable adhesive disposed between said layers.

7. The bioengineered construct of claim 6, wherein the bioremodelable or bioresorbable adhesive is a solution derived from Bombyx mori silkworm.

8. The bioengineered construct of claim 7, wherein the solution comprises silk fibroin at a concentration between about 2% to about 8% w/v.

9. The bioengineered construct of claim 2, wherein at least one layer of extracellular matrix is crosslinked.

10. The bioengineered construct of claim 2, wherein at least one layer of extracellular matrix is crosslinked to a lesser degree and at least one layer of extracellular matrix is crosslinked to a higher degree.

11. The bioengineered construct of claim 1, wherein the layer is produced in conditions that include a chemically defined culture medium containing no animal-derived components.

12. The bioengineered construct of claim 1, wherein the extracellular matrix producing cells are derived from tissue selected from the group consisting of neonate male foreskin, dermis, tendon, lung, urethra, umbilical cord, corneal stroma, oral mucosa, and intestine.

13. The bioengineered construct of claim 1, wherein the extracellular matrix producing cells are derived from stem cells.

14. The bioengineered tissue construct of claim 1, wherein the cultured extracellular matrix producing cells are dermal fibroblasts.

15. A method for making a bioengineered construct, comprising:
culturing extracellular matrix producing cells in a first culture under conditions that induce the cells to form a first layer of extracellular matrix;
culturing extracellular matrix producing cells in a second culture under conditions that induce the cells to form a second layer of extracellular matrix;
terminating the extracellular matrix producing cells in both the first layer of extracellular matrix and second layer of extracellular matrix to form first and second devitalized layers of extracellular matrix;
contacting the first devitalized layer of extracellular matrix to the second devitalized layer of extracellular matrix by superimposing said first and second layers to form a bonding region;
bonding said first and second layers, wherein said bonding is achieved by crosslinking or an adhesive.
